(19) United States
(12) Patent Application Publication
(10) Pub. No.: US 2026/0077052 A1
(43) Pub. Date: Mar. 19, 2026

(54) Methods For Reducing Or Preventing SARS-COV-2 Infection And Transmission

(71) Applicant: Decoy Therapeutics Inc., Cambridge, MA (US)

(72) Inventors: Barbara L. HIBNER, Winchester, MA (US); Jodi COOPER, San Diego, CA (US)

(21) Appl. No.: 19/195,229

(22) Filed: Apr. 30, 2025

Related U.S. Application Data
(60) Provisional application No. 63/640,320, filed on Apr. 30, 2024.
(60) Provisional application No. 63/776,427, filed on Mar. 24, 2025.

(51) Int. Cl. A61K 47/65; A61K 47/54; A61P 31/14

(52) U.S. Cl. CPC A61K 47/65; A61K 47/554; A61P 31/14

(57) ABSTRACT

The application provides a method to prevent or reduce the transmission of a coronavirus, such as a SARS-COV-2 variant, or a paramyxovirus from an infected subject to other uninfected subjects, comprising administrating an anti-viral peptide conjugate to the infected subject, the uninfected subject, or both.

### RELATED APPLICATIONS

This application claims the benefit of U.S. Provisional Application No. 63/776,427, filed on Mar. 24, 2025 and U.S. Provisional Application No. 63/640,320, filed on Apr. 30, 2024. The entire teachings of the above applications are incorporated herein by reference.

### BACKGROUND OF THE INVENTION

Severe Acute Respiratory Syndrome Coronavirus 2 (SARS-Cov-2) is a coronavirus that emerged in late 2019, leading to the global COVID-19 pandemic. It is the causative agent of COVID-19, a respiratory illness that can range from mild or asymptomatic cases to severe pneumonia and even death.

One of the primary factors fueling the global pandemic and the ongoing impact of SARS-Cov-2 on our daily lives is its exceptional transmissibility. SARS-COV-2 is primarily transmitted through respiratory droplets released when an infected person talks, coughs, sneezes, or even breathes. These droplets, laden with viral particles, can be inhaled by individuals in close proximity, typically within about six feet (two meters). Moreover, SARS-COV-2 can also spread through aerosols, smaller respiratory particles that can linger in the air for extended periods. In indoor settings with poor ventilation, these aerosols pose a heightened risk, potentially reaching individuals further away from the infected person. The virus can even survive on surfaces for varying amounts of time, depending on the material. When individuals touch contaminated surfaces and then touch their face, particularly their mouth, nose, or eyes, they can introduce the virus into their respiratory system. Close and prolonged contact with an infected person, especially in indoor environments, heightens the transmission risk. Crowded spaces and inadequate ventilation can foster super-spreader events, leading to rapid transmission and significant outbreaks among large groups of people.

Efforts to curtail transmission typically involve isolating infected individuals until they test negative for SARS-COV-2, requiring them to remain separated from family members and away from schools or workplaces for several days. While this approach can reduce transmission, it places a significant physical and emotional burden on infected individuals and their caregivers, and it can hinder social efficiency, especially given the prevalence of SARS-COV-2 infections.

Paramyxoviridae is a family of negative-strand RNA viruses. These viruses are found in vertebrates and cause diseases such as measles, mumps, and respiratory tract infections.

The Paramyxoviridae viral family includes established human pathogens such as measles virus, mumps virus, and the human parainfluenza viruses (HPIVs); highly lethal zoonotic pathogens such as Nipah virus; and a number of recently identified agents, such as Sosuga virus. Paramyxoviruses are members of the order Mononegavirales and have nonsegmented, negative-sense RNA genomes encapsidated into a ribonucleoprotein (RNP) complex within an enveloped virion. All paramyxovirus genomes encode for a nucleocapsid (N) protein, a phospho-(P) protein, a matrix (M) protein, a fusion (F) glycoprotein, an attachment hemagglutinin (H)/hemagglutinin-neuraminidase (HN)/glyco-(G) protein, and an RNA-dependent RNA polymerase, or large (L) protein. In addition, some paramyxoviruses encode a small hydrophobic protein and RNA editing of the P protein gene leads to expression of additional nonstructural proteins that play key roles in the antiviral response.

The high human-to-human transmission rate of paramyxoviruses such as measles virus and the high case fatality rate associated with other family members such as Nipah virus are of concern. Effective live-attenuated vaccines and a subunit vaccine have been developed for a small subset of paramyxoviruses, and these have been used to limit geographical distribution and eradicate important human and veterinary diseases. However, no effective approved antiviral treatments or vaccines exist to mitigate the majority of paramyxoviral diseases and we are woefully unprepared should a respiratory paramyxovirus emerge and spread in humans with no preexisting immunity.

For example, measles is a highly contagious viral infection that can cause serious health complications, especially in children younger than 5 years old. Measles is caused by a single-stranded, enveloped RNA virus with 1 serotype, which is classified as a member of the genus Morbillivirus in the Paramyxoviridae family. Despite measles being declared eliminated from the United States in 2000, measles is still common in many parts of the world and is brought into the United States from other countries.

Therefore, there is a pressing need for more effective methods that can prevent SARS-CoV-2 and paramyxoviridae viral transmission while affording infected individuals the opportunity to lead normal lives.

### SUMMARY OF THE INVENTION

The present invention is based on the discovery of a novel method to effectively prevent or reduce transmission of a SARS-Cov-2 variant from an infected individual to other individuals, thereby preventing or reducing subsequent infections. The method comprises administrating a peptide conjugate to the infected individual, preferably via intranasal administration such as using an intranasal spray, an inhaler or a nebulizer.

The present invention is also based on the discovery that the peptide conjugate of the invention can be administered to effectively prevent or reduce transmission of a paramyxovirus, thereby preventing or reducing subsequent infections. The peptide conjugate is administered to the infected individual, preferably via intranasal administration such as using an intranasal spray, an inhaler or a nebulizer. In embodiments, the paramyxovirus is selected from the measles virus, the mumps virus, the human parainfluenza virus (HPIV) or the Nipah virus. Preferably, the paramyxovirus is the measles virus. Preferably, the paramyxovirus is the mumps virus. Preferably, the paramyxovirus is the human parainfluenza virus (HPIV). Preferably, the paramyxovirus is the Nipah virus.

The peptide conjugate is represented by: (Peptide-Linker) n-B-Hydrophobic Moiety, wherein each Peptide is independently a short amino acid sequence, preferably a therapeutic peptide, such as an HRC Peptide or a targeting peptide, each Linker is independently optional and the Peptide is a short amino acid sequence, preferably a therapeutic peptide, B is a multivalent moiety, and n is an integer selected from 1, 2, 3 or more. Hydrophobic Moiety is preferably a membrane integrating lipid.

Also provided is a method to prevent or reduce infection associated with a SARS-Cov-2 variant in a subject in need.

Also provided is a method to prevent or reduce paramyxovirus viral infection in a subject in need.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a schematic diagram of the study design of the transmission study described in Examples.

FIG. 2 shows the transmission study results: (a) RT-qPCR is a terminal measurement, which measures live and dead viral genomes; (b) TCID₅₀ is a terminal measurement, which measures live virus; and (c) body weight change measured from start of experiment to day experiment ended.

FIG. 3 shows efficacy evaluation based on viral RNA measured by throat swabs on (a) Day 2, (b) Day 3, and (c) Day 4.

FIG. 4 shows lung histology results reflected in terms of cumulative lung score as defined herein.

FIG. 5A through FIG. 5E show alpha fold predicted structures of viral HR1 and HR2 six helical bundles for the HR1 and HR2 sequences from Tables 3 and 4, respectively, and an overlay of the bundles is shown.

FIG. 6 shows the ligand binding (EC₅₀ (μM)) to certain viral proteins. The ligands are conjugated to cholesterol as disclosed in PCT/US24/31869, filed on May 31, 2024, with the structure of Peptide Conjugate 12.

FIG. 7 shows the MMGBSA binding energy (kcal/mole) for each conjugated ligand with HR1 proteins of measles, Nipah and HPIV3.

### DETAILED DESCRIPTION OF THE INVENTION

### Method of the Invention

Since its emergence in 2019, SARS-Cov-2 with its exceptional transmissibility rapidly sparked a global pandemic that had persisted for over two years, affecting people in the U.S. and all over the world. Despite relentless efforts to combat the virus, SARS-COV-2 has undergone numerous evolutions, leading to the emergence of variants that continue to impact our daily lives. When individuals become infected with a SARS-COV-2 variant, the standard approach requires adults to stay away from workplaces, children to stay away from schools, and everyone to isolate from family members for several days or until they test negative. However, given the widespread prevalence of SARS-COV-2 infections, this physical separation strategy, while effective in curbing transmission, exacts a substantial toll on social productivity and individuals' psychological and mental well-being.

This invention presents a novel method designed to effectively prevent or reduce the transmission of a coronavirus or variant thereof (preferably a SARS-COV-2 variant) from an infected subject to other otherwise uninfected subjects, even when close contact occurs between the infected and uninfected groups. Consequently, it offers individuals an opportunity to maintain their normal daily activities in the event of a virus infection or contact with an infected person.

This invention presents a method designed to effectively prevent or reduce the transmission of paramyxovirus from an infected subject to other otherwise uninfected subjects, even when close contact occurs between the infected and uninfected groups. Consequently, it offers individuals an opportunity to maintain their normal daily activities in the event of a virus infection or contact with an infected person.

Preferably, the paramyxovirus is the measles virus. Preferably, the paramyxovirus is the mumps virus. Preferably, the paramyxovirus is the human parainfluenza virus (HPIV). Preferably, the paramyxovirus is the Nipah virus.

The term “preventing or reducing transmission” as used herein refers to a variety of situations where, in contact with the infected subject, the uninfected subject remains negative for covid or paramyxovirus; the uninfected subject does not show symptoms of infection or only shows minor symptoms; the uninfected subject remains negative for covid or paramyxovirus and does not show symptoms of infection or only shows minor symptoms; and the uninfected subject does not show symptoms of infection or only shows minor symptoms not withstanding a positive test result.

The term “contact” as used herein means direct or indirect physical interaction or exposure between an infected subject (the source) and a susceptible subject (the recipient) that can result in the transfer of the virus from the infected subject to the uninfected subject. It is meant to include the act of occupying a shared space (such as a residence, workplace, classroom, car, a concert, or any enclosed area) concurrently, or within a brief time frame (e.g., the uninfected subject enters the space within about 24 hours, about 48 hours, or about 72 hours after the infected subject leaves the space).

The method comprises administrating an effective amount of peptide conjugate to a subject infected with a coronavirus or variant thereof or a paramyxovirus, wherein the peptide conjugate is represented by:

(Peptide-Linker)ₙ-B-Hydrophobic Moiety,
wherein the Linker is independently optional and independently a bivalent linking moiety, each Peptide is independently a short amino acid sequence, a therapeutic peptide, such as a HRC peptide derived from a coronavirus spike protein or a paramyxovirus fusion protein, B is a multimeric core which covalently links each peptide moiety to the hydrophobic moiety, and n is an integer selected from 1, 2, 3 or more. Hydrophobic moiety has a function of anchoring to the cell membrane and can be for example a membrane integrating lipid.

The term “infected” as used herein refers to the incident wherein a subject or organism that has been exposed to the virus and has had the virus enter their body, where it can potentially multiply and cause illness. Infection with for example SARS-COV-2 can lead to a wide range of symptoms, from mild or asymptomatic cases to severe respiratory distress and other complications. In some embodiments, the methods of the invention treat, prevent, or ameliorate a SARS-COV-2 (COVID-19) respiratory infection.

Paramyxovirus infection can also lead to a wide range of symptoms, from mild cases to severe respiratory distress and other complications. For example, infection with measles can lead to a wide range of symptoms, such as a high fever (may spike to more than 104° F.), cough, runny nose (coryza), red, watery eyes (conjunctivitis), and a rash. Measles can cause serious health complications, especially in children younger than 5 years of age. Common complications are ear infections and diarrhea. Serious complications include pneumonia and encephalitis. In some embodiments, the methods of the invention treat, prevent, or ameliorate a measles infection.

The term “transmission” used as herein refers to the process by which SARS-COV-2 is passed from an infected subject to an otherwise uninfected subject, resulting in the viral infection of the otherwise uninfected subject. The term “transmission” also refers to the process by which paramyxovirus is passed from an infected subject to an otherwise uninfected subject, resulting in the viral infection of the otherwise uninfected subject.

The SARS-Cov-2 variant can comprise one or more mutations in the viral fusion protein with reference to the wild type. The SARS-Cov-2 variant can comprise at least 1, at least 2, at least 3, at least 4, at least 5 mutations. In some embodiments, the variant comprises at least 10 mutations. In some embodiments, the variant comprises at least 15 mutations. In some embodiments, the variant comprises at least 20 mutations.

In some embodiments, the SARS-Cov-2 variant comprises at least 5 mutations wherein the at least 5 mutations are independently in the spike protein S1 subunit or the S2 subunit or combinations thereof.

In some embodiments, the at least 5 mutations are independently in N-Terminal domain (NTD), the receptor binding domain (RBD), the fusion peptide (FP) domain, the heptad repeat 1 (HR1) domain, or combinations thereof.

In some embodiments, the at least 5 mutations are independently selected from at least 5 mutations from the SAR-Cov-2 Alpha variant; at least 5 mutations from the SAR-Cov-2 Beta variant; at least 5 mutations from the SAR-Cov-2 Delta variant; or at least 5 mutations from the SAR-Cov-2 Omicron variant. In some embodiments, the at least 5 mutations are independently selected from at least 5 mutations from the SAR-Cov-2 Alpha variant. In some embodiments, the at least 5 mutations are independently selected from at least 5 mutations from the SAR-Cov-2 Beta variant. In some embodiments, the at least 5 mutations are independently selected from at least 5 mutations from the SAR-Cov-2 Delta variant. In some embodiments, the at least 5 mutations are independently selected from at least 5 mutations from the SAR-Cov-2 Omicron variant.

In some embodiments, the SARS-COV-2 variant comprises at least one variant selected from B.1.1.7 (Alpha), B.1.351 (Beta), P.1 (Gamma), B.1.617.2 (Delta), B.1.429/B.1.427 (Epsilon), B.1.617.1 (Kappa), B.1.525 (Eta), B.1.526 (Iota), P.3 (Theta), P.2 (Zeta), and B.1.1.529 (Omicron).

In some embodiments, the SARS-COV-2 variant comprises at least one variant selected from A.1-A.6, B.3-B.7, B.9, B.10, B.13-B.16, B.2, B.1 lineage, P.1, P.2, P.3, and R.1.

In some embodiments, the B.1 lineage comprises at least one of (including, but not limited to, B.1, B.1.1, B.1.1.7, B.1.1.7 with E484K, B.1.2, B.1.5-B.1.72, B.1.9, B. 1.13, B. 1.22, B.1.26, B.1.37, B.1.3-B.1.66, B.1.177, B.1.243, B.1.313, B.1.351, B.1.427, B.1.429, B.1.525, B.1.526, B.1.526.1, B.1.526.2, B.1.617, B.1.617.1, B.1.617.2, B.1.617.3, B.1.619, B.1.620, and B.1.621.

In some embodiments, the peptide conjugate of the invention is administered to a subject in need via nasal administration such as nasal spray, nasal drops, nasal gels, nasal powders, nasal aerosols, nasal pumps, nasal nebulizers, nasal inhalers. Preferably the nasal administration is achieved using an intranasal spray, an inhaler, or a nebulizer.

In some embodiments, the peptide conjugate of the invention is administered to a subject in need through pulmonary drug delivery, in which patients preferably use an inhaler to inhale a pharmaceutical composition comprising the peptide conjugates and the peptide conjugates are absorbed into the bloodstream via the lung mucous membrane. An inhaler includes all available types of inhalers on the market, such as metered-dose inhalers (MDI), dry powder inhalers (DPI), soft mist inhalers (SMI) and nebulizers (e.g., a jet nebulizer, an ultrasonic wave nebulizer).

In some embodiments, the peptide conjugate is administered in combination with at least one other antiviral active agent or therapy.

The infected subject, preferably a human, can be an individual diagnosed with the infection and is either symptomatic, pre-symptomatic, or asymptomatic, or at risk for developing infection. For example, the subject can be at risk for developing the viral respiratory infection due to direct or indirect exposure or possible exposure to the virus (such as SARS-COV-2 or a mutant thereof or a paramyxovirus), such as via exposure to an infected individual or a virus-contaminated fomite. The subject can be a resident of, or a visitor to, a community in which the viral respiratory infection has been identified, for example, the subject can be a family member of an infected individual or the subject can work in a health care setting caring for infected individuals. In some embodiments, the subject at risk for infection is asymptomatic and has tested negative for presence of the virus prior to the commencement of therapy. In specific examples, the subject can be at risk for developing COVID-19 due to exposure to the SARS-CoV-2 virus, for example, from the respiratory droplets or aerosols of an infected individual and/or contact with a contaminated fomite. In yet further aspects, the subject is suffering from COVID-19 including subjects suffering from mild, moderate, or severe COVID-19. In yet further aspects, the subject is suffering from paramyxovirus, for example, measles.

In some cases, the peptide conjugate can be designed to serve as a pan-inhibitor, capable of targeting multiple related viruses or proteins across a viral family or protein class. For example, the conjugate may incorporate peptides that collectively inhibit a broad spectrum of paramyxoviruses, coronaviruses, or other viral groups by engaging conserved regions critical for viral entry or replication (preferably paramyxoviruses, or coronaviruses).

In some cases, the peptide conjugate is engineered to function as a selective inhibitor, wherein the conjugated peptides are specifically tailored to block a particular virus or biological target with high specificity. Such selective conjugates may enhance antiviral potency, improve pharmacokinetic properties, extend serum half-life, promote multivalent binding to target sites, or enable targeted delivery to specific tissues or cell types.

In some cases, the peptide conjugate comprises peptides that act as pan-paramyxovirus inhibitors, blocking a range of viruses including measles virus, mumps virus, parainfluenza (HIPV3) viruses, and Nipah virus by targeting conserved fusion machinery or other essential viral components.

In some cases, the peptide conjugate comprises peptides that act as pan-coronavirus inhibitors, capable of targeting SARS-COV, MERS-COV, SARS-COV-2, and related emerging coronaviruses by interfering with conserved regions of the spike protein, preferably the HR1/HR2 domains.

In some cases, the peptide conjugate comprises selective inhibitors targeting the same virus, such as peptides that are individually selective for measles virus, mumps virus, parainfluenza virus, Nipah virus, MERS-COV, or SARS-COV-2, respectively. The use of highly selective peptides may reduce off-target effects and maximize therapeutic precision.

In some cases, while the individual peptides of the invention are selective for specific viruses, a peptide conjugate comprising two or more different selective peptides can function collectively as a pan-inhibitor. For example, a conjugate combining a measles virus-selective peptide with a mumps virus-selective peptide and a parainfluenza virus-selective peptide may exhibit broad-spectrum activity against the paramyxovirus family as a whole.

In some embodiments of the method of the invention, the infected subject suffers from another disease or condition, such as chronic obstructive pulmonary disease (COPD) or ulcerative colitis, which can be exacerbated by an infection.

The peptide conjugate is administered to the infected subject upon the discovery of the infection, before infection as a prophylactic measure, or within 72 hours of the discovery of the infection. The peptide conjugate is preferably administered to the infected subject before the infected subject comes into contact with other uninfected subjects. The peptide conjugate is preferably administered to the infected subject within 48 hours, 36 hours, 24 hours, 12 hours, or 8 hours of the discovery of the infection, or 48 hours, 36 hours, 24 hours, 12 hours, or 8 hours before the infected subject comes into contact with other uninfected subjects. Preferably the administration continues until the infected subject test negative for the coronavirus or a paramyxovirus, such as measles. Contact may involve sharing a residence, workplace, classroom, car, or any enclosed area. It may also involve participating in a gathering or event, such as a concert. The term “enclosed area” as used herein refers to any space that is enclosed or substantially enclosed by physical barriers, such as walls, fences, doors, or other structures. Enclosed areas can vary widely in size and purpose, ranging from small rooms or compartments to large buildings and structures.

In additional embodiments, the method further comprises an optional step of administering an effective amount of peptide conjugate to the other uninfected subject(s) before the uninfected subject(s) come into contact with the infected subject. In some cases, the method further comprises an optional step of administering an effective amount of peptide conjugate to the other uninfected subject(s) 48 hours, 36 hours, 24 hours, 12 hours, 8 hours before the uninfected subject(s) come into contact with the infected subject. In some cases, the administration to the uninfected subject(s) continues throughout the duration of physical contact with the infected subject or until the infected subject tests negative, whichever occurs first.

In alternative embodiments, provided is also a method for preventing or reducing the transmission of a coronavirus or variant thereof (preferably a SARS-COV-2 variant) from an infected subject to other otherwise uninfected subjects when in contact with the infected subject, comprising administrating an effective amount of the peptide conjugate as described herein to the uninfected subject. The peptide conjugate is preferably administered to the uninfected subject 48 hours, 36 hours, 24 hours, 12 hours, 8 hours before the uninfected subject(s) come into contact with the infected subject. Preferably the administration continues throughout the duration of physical contact with the infected subject or until the infected subject tests negative, whichever occurs first.

In alternative embodiments, provided is also a method for preventing or reducing the transmission of paramyxovirus from an infected subject to other otherwise uninfected subjects when in contact with the infected subject, comprising administrating an effective amount of the peptide conjugate as described herein to the uninfected subject. The peptide conjugate is preferably administered to the uninfected subject 48 hours, 36 hours, 24 hours, 12 hours, 8 hours before the uninfected subject(s) come into contact with the infected subject. Preferably the administration continues throughout the duration of physical contact with the infected subject or until the infected subject tests negative, whichever occurs first.

The peptide conjugate is preferably administered in a pharmaceutical composition to the infected subject before the infected subject is symptomatic (e.g., pre-symptomatic), at the onset of symptoms, or within 24 hours of the onset of symptoms. The pharmaceutical composition can be administered at a variety of dosing schedules. For example, the pharmaceutical composition can be administered one or more times and over a course of one or more days. In some embodiments, the pharmaceutical composition is administered one or more times per day for one to 10 days. In some embodiments, the pharmaceutical composition is administered one or more times per day until the subject is asymptomatic and/or testing for the virus is negative.

The pharmaceutical composition can be administered to the nasal passages using routine methods and devices (see D. Marx et al., IntechOpen, DOI: 10.5772/59468. Available on the world wide web at intechopen.com/books/drug-discovery-and-development-from-molecules-to-medicine/intranasal-drug-administration-an-attractive-delivery-route-for-some-drugs). For example, the pharmaceutical composition can be administered to the nasal passages as drops or as an aerosol spray, for example, using an aerosol bottle or a multi-dose spray pump, which can provide a uniform metered dose. The volume per dose can be varied, but is typically from about 50 to about 150 μl. The desired volume will depend on the desired dose of the active agent and the concentration of the active agent in the composition.

In some embodiments, the pharmaceutical composition of the invention is formulated for nebulization. The peptide conjugate of the invention is administered to a subject in need by nebulization through a nebulizer. A nebulizer is used to deliver medication in the form of an aerosolized mist inhaled into the lungs. The medication formulation is aerosolized by compressed gas, or by ultrasonic waves. A jet nebulizer can be connected to a compressor. The compressor emits compressed gas through a liquid medication formulation at a high velocity, causing the medication formulation to aerosolize. Acrosolized medication may be then inhaled by the patient. An ultrasonic wave nebulizer generates a high frequency ultrasonic wave, causing the vibration of an internal element in contact with a liquid reservoir of the medication formulation, which causes the medication formulation to aerosolize. Aerosolized medication may be then inhaled by the patient. A nebulizer may utilize a flow rate of between about 3-12 L/min, such as about 6 L/min. In some examples, the milled active may be suspended in a pharmaceutically acceptable liquid carrier vehicle and administered by nebulization (e.g., air jet nebulization). A nebulizer treatment takes about 3 to about 30 minutes to complete, about 3 to about 20 minutes, or about 3 to about 15 minutes.

Where delivery to the pulmonary system, or lungs, is desired, it can be efficacious to acrosolize a low concentration solution of the active agent for an extended period, such as overnight.

In addition to the prevention or reduction of viral transmission from the infected subject to other uninfected subject(s), the method as described above also has a therapeutic effect on the treatment of the infected subject.

Provided is also a method of treating infection associated with a SARS-Cov-2 variant in a subject in need, wherein the method comprises administering an effective amount of the peptide conjugate as described herein.

Provided is also a method of treating infection associated with paramyxovirus in a subject in need, wherein the method comprises administering an effective amount of the peptide conjugate as described herein. In embodiments, the paramyxovirus is selected from the measles virus, the mumps virus, the human parainfluenza virus (HPIV) or the Nipah virus. Preferably, the paramyxovirus is the measles virus. Preferably, the paramyxovirus is the mumps virus. Preferably, the paramyxovirus is the human parainfluenza virus (HPIV). Preferably, the paramyxovirus is the Nipah virus.

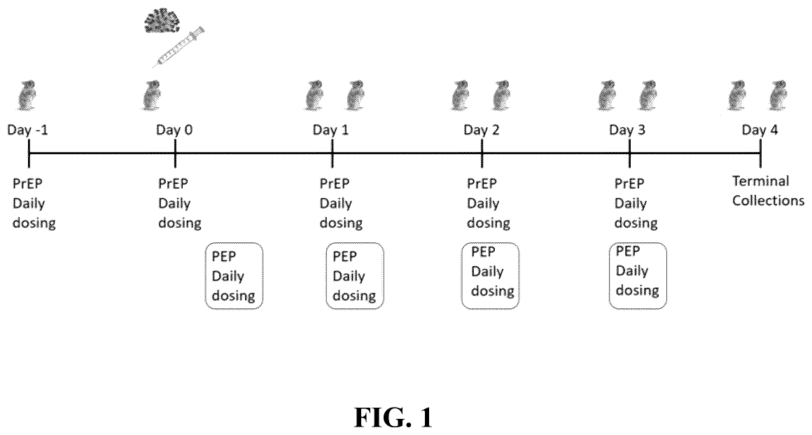

The method of the invention is studied using an animal model. The animal model is susceptible to SARS-COV-2 or a variant thereof and preferably exhibits similar disease characteristics to humans, based on their species, age, and genetic background. An animal model includes mice, rat, hamster, Guinea pig, ferret, and non-human primates (such as monkey). In some embodiments, the animal model is a hamster or ferret. The animals are divided into multiple treatment groups, including pre-exposure prophylaxis (PrEP) groups, post-exposure prophylaxis (PEP) groups, and appropriate control groups. Each group should be further divided into donor and contact subgroups, where the donor animals are infected and the contact groups are contacted with the donor, preferably by cohousing. The animals are housed in appropriate environmental conditions conducive to viral transmission studies. Implemented cohousing conditions are designed to facilitate direct contact between the donor and contact animals within each treatment group. Before cohousing, the donor animals are anesthetized (e.g., intraperitoneally using Ketamin (60 mg/kg) and Xylazin (6 mg/kg)) and inoculated intranasally with SARS-COV-2 or a variant thereof (e.g., SARS-COV-2 delta variant). For treatment to prevent transmission, the donor animals, the contact animals, or both, are administered with the peptide conjugate of the invention (preferably intranasally). The administration is once daily, twice daily, three times daily, once every 36 hours, once 48 hours, according to the specified dosage (such as about 1 mg/kg, about 5 mg/kg, about 10 mg/kg, about 15 mg/kg, about 20 mg/kg of the peptide conjugate). Preferable administration and dosage are once daily and about 10 mg/kg of the peptide conjugate. The animals are monitored closely for any adverse reactions to the treatment. Preferably, the animals are monitored daily for changes in appearance, behavior, and weight throughout the duration of the study. Clinical signs of disease progression or treatment efficacy are recorded daily. Throat swabs from the animals are collected at specified time points post-infection for viral quantification using RT-qPCR. The cohousing and monitoring period of donor and contact animals is at least three days (four, five, six, seven, eight, nine, ten, or more than ten days). The animals at the designated endpoint are euthanized using an approved euthanasia method. Tissues, preferably lungs, are collected for viral quantification (e.g., using RT-qPCR) and histopathological analysis (e.g., using H&E staining) at the endpoint. Additional tissues are optionally collected for viral quantification and/or histopathological analysis. For example, nasal concha (also called nasal turbinate or turbinal), as a major site of viral replication, is collected for viral quantification and histopathological analysis. In some embodiments, certain groups of animals are euthanized at the midpoint of the experiment and tissues collected for viral quantification and/or histopathological analysis. FIG. 1 provides one example of animal study design.

### Peptide

Coronaviruses target human cells via the spike protein binding domain (RBD) attaching to the human angiotensin converting enzyme 2 (hACE2) receptor on host cells. The coronavirus spike (S) glycoprotein is a class I viral fusion protein on the outer envelope of the virion that plays a critical role in viral infection by recognizing host cell receptors and mediating fusion of the viral and cellular membranes. Coronavirus entry into host cells is mediated by the transmembrane spike (S) glycoprotein that forms homotrimers protruding from the viral surface. S comprises two functional subunits responsible for binding to the host cell receptor (S1 subunit) and fusion of the viral and cellular membranes (S2 subunit).

S1 serves the function of receptor-binding and contains a signal peptide (SP) at the N terminus, an N-terminal domain (NTD), and receptor-binding domain (RBD). S2 functions in membrane fusion to facilitate cell entry, and it contains a fusion peptide (FP) domain, internal fusion peptide (IFP), two heptad-repeat domains (HR 1 and HR2), transmembrane domain, and a C-terminal domain.

After binding, the spike protein is activated by the host cell transmembrane protease/serine subfamily member 2 (TMPRSS2) and consequently the virus undergoes fusion with the endosomal membrane for entry into the cell. The membrane fusion domain of the spike protein in coronaviruses is highly conserved so targeting membrane fusion may result in durable long-lasting therapeutics. The mechanisms of the viral entry have been illustrated in Jackson et al, “Mechanisms of SARS-COV-2 entry into cells.” Nat Rev Mol Cell Biol 23, 3-20 (2022). https://doi.org/10.1038/s41580-021-00418-x.

Each peptide is independently a short amino acid sequence, including therapeutic peptides for coronavirus or peptide inhibitors inhibiting coronavirus (such as peptide inhibitors inhibiting the fusion process). In some embodiments, each Peptide independently has a length ranging from 5 to 100 amino acid residues, from 6 to 80 amino acid residues, from 8 to 60 amino acid residues, or from 10 to 50 amino acid residues. In preferred embodiments, each Peptide independently has a length of 12-40 amino acid residues. In preferred embodiments, each Peptide independently has a length of 18-39 amino acid residues.

In some embodiments, each Peptide is independently derived from a coronavirus spike fusion protein and/or target protein, preferably from C-terminus heptad repeat (HRC) region of a spike fusion protein. The HRC region of the coronavirus spike fusion protein is involved in the formation of the six-helix bundle structure that drives viral fusion. HRC peptides mimic the HRC region and can bind to the N-terminal heptad repeat (HRN) region of the fusion protein, blocking the formation of the six-helix bundle and preventing viral fusion and entry into host cells. In some embodiments, a HRC peptide is a wild type peptide derived from the HRC region of a coronavirus spike fusion protein. In other embodiments, an HRC peptide is a mutant or variant peptide derived from the HRC region of a coronavirus spike fusion protein, which can comprise genetic mutations that occur in nature, and/or modifications made in a laboratory setting.

In some embodiments, each peptide is independently a HRC peptide derived from a coronavirus spike protein and/or a targeting peptide, provided that there is at least one HRC peptide derived from a coronavirus spike protein. While preferred embodiments of the invention utilize native or wild-type peptides, non-natural peptides can be used as well. For example, amino acids found in one or more mutations (e.g., omicron mutations) can be combined with the native sequences of other viruses (e.g., the delta virus). The so-called HRC peptide or region of the coronavirus spike protein is preferred. The HRC peptides inhibit viral fusion, an important 5 early step in the infection process. The wild type HRC peptide is a conserved region of the spike, or S, protein across coronaviruses. The conserved nature of the fusion regions (HRC/HRN) and mechanism of the class I enveloped viruses make it an ideal target to develop a pan-coronavirus inhibitor.

Preferred wild type HRC peptides comprise the sequence and binding fragments thereof:

(SEQ ID NO. 1)
Acₙ-DISGINASVVNIQKEIDRLNEVAKNLNESLIDLQEL,

wherein n is 0 or 1.

With regard to the HRC peptide in SEQ ID NO.1, the conventional numbering of the amino acids begins with 1168 at D. 1169I, 1172I and 1176V are in the hydrophobic interface pre-fusion and 1177V is exposed. These amino acids stabilize a helix. When the conformation change occurs (e.g., protease clipping to release FP), 1176V is exposed and 1177 V presents in the hydrophobic surface interacting with HRN trimer. 1173N, and 1194N are implicated in N-linked glycosylation conserved in coronavirus. The first 7 amino acids are implicated in HRN binding. The “N-Cap” region spans 1177V and 1189V. The 1189V is a conserved hydrophobe in coronaviruses and stabilizes the HRC hydro-core and is involved in the HRN interaction.

The hydrophobic core spans 1179I and 1193L and is helical pre- and post-fusion. The isoleucines, leucines and alanine are important in folding and stability of a coiled coil. The C-Cap region spans 1194N and 1203L, 1197L, 1200L and 1203L are in the hydrophobic interface pre-fusion and 11801 is exposed. These amino acids stabilize the helix. 1203L may be implicated in hydrophobic packing between three polypeptide chains in a trimeric coiled coil. When confirmation change occurs (protease clipping to release FP), 1197L is exposed and 1198I is in the hydrophobic surface interacting with a HRN trimer. 1182E and 1202E form a salt bridge between HRC and HRN. Further, 1177V, 1178N, 1189V and 1198I have been shown to interact with HRN in crystal structures.

In some embodiments, the peptides are selected from variants of a wild type HRC peptides comprising the sequence and binding fragments thereof:

(SEQ ID NO. 1)
Acₙ-DISGINASVVNIQKEIDRLNEVAKNLNESLIDLQEL,

wherein n is 0 or 1.

In the context of protein variants, the term “variant” is defined as a peptide which has at least one amino acid deleted, added, or substituted in comparison with a wild type sequence, such as SEQ ID NO. 1 or other native sequence described herein. Variants preferably bind the cognate ligand of the wild type sequence. For example, a peptide wherein 1, 2, 3, 4 or 5 amino acids of SEQ ID NO. 1 are substituted can be used. Such substituted amino acids can preferably be selected from one or more corresponding amino acids identified in a different coronavirus strain via a sequence alignment, such as shown above. For example, one or both underlined isoleucines can be substituted with leucine and/or methionine, as described in the alignment provided above. The underlined alanine can be substituted by valine, leucine or isoleucine. One or both underlined leucines can be independently substituted by isoleucine, tyrosine, alanine or valine. Other conservative or nonconservative substitutions, (lysine and glutamine or aspartic acid and glutamic acid) can be selected as well. In some embodiments, amino acids that are conserved amongst 2, 3, 4, 5 or more coronavirus (e.g., coronavirus isolated from bats or SARS-CoV2 mutants or variants) remain conserved in the non-natural HRC peptide.

For example, the wild type HRC sequence can include 1, 2, 3, 4, 5, 6, 7, 8, 9, 10 or more additional amino acids native to the S protein at the N- and/or C termini. For example, glycine can be added to the N-terminus. Additionally, the wild type HRC peptide fragment can delete 1, 2, 3, 4, 5, 6, 7, 8, 9, or 10 amino acids at the N and/or C termini and inhibit infection. Typically, not more than ten total amino acids are deleted in total. For example, the 10 amino acids at the C terminus can be deleted and be expected to retain inhibitory activity.

In some embodiments, modifications to wild type sequences are desirable. For example, using one or more D amino acids can improve pharmacokinetics and the half-life of the peptide. Thus, in some embodiments, the invention includes peptides characterized by 1, 2, 3, 4, 5, 6, 7, 8, 9, 10 or more D-amino acids. The D-amino acids can preferably be a corresponding L-amino acid of the wild type sequence. In some embodiments, the D-amino acid is an amino acid located at or near (e.g., within 1, 2, or 3 amino acids) a protease degradation site. In some embodiments, the D-amino acid is a hydrophobic amino acid participating in binding with the HRN peptide and preferably at a higher affinity than the corresponding wild type sequence. Alternatively, or additionally, the D-amino acid is a hydrophilic amino acid, such as lysine, aspartic acid, glutamic acid or arginine. Alternatively, or additionally, the D-amino acid can be selected from the 7 amino acids at the N-terminus of SEQ ID NO:1. Peptides that have been improved by incorporating D-amino acids are described in U.S. Ser. No. 63/140,387, filed on Jan. 22, 2021, which is incorporated by reference in its entirety.

However, swapping one or more D-amino acids for the corresponding L-amino acid can change the topology of the peptide and impact function. Therefore, a preferred non-natural HRC peptide is a Retro-Inversion HRC peptide, or “RI HRC peptide”. Retro-Inversion HRC peptides are preferably characterized by a binding affinity of at least about 50% of the wild-type HRC peptide with its cognate ligand in a standard binding assay and decreased susceptibility to mammalian protease degradation. Retroinversion is defined as reversing a D-peptide sequence of a helical peptide or “flipping” the termini thereby restoring the presentation of the side chains to the binding ligand or target. See Kim et al, Method to generate highly stable D-amino acid analogs of bioactive helical peptides using a mirror image of the entire PDB, PNAS, Feb. 13, 2018, 115 (7) 1505-1510, which is incorporated herein by reference in its entirety. Therefore, a non-natural peptide of the invention can include a peptide having the sequence of SEQ ID NO:1 wherein amino acids are D-amino acids, such as the amino acids within a region, flipping the N-terminus for a C terminus. For example, the N termini can be subjected to retroinversion as shown in SEQ ID NO. 2 where each D amino acid is preceded by a “d”:

(SEQ ID NO. 2)
dIdGdSdIdD NASVVNIQKEIDRLNEVAKNLNESLIDLQEL.

This example offers a single RI region of 5 amino acids. However, as few as two amino acids can be selected (e.g., the 2 N-terminal amino acids). For example, the RI region can span the hydrophobic core, 1178N to 1194N, or the C-cap region or a portion thereof. Alternatively, the entire peptide can be an RI peptide. Additionally, two, three or more RI regions can be included. For example, both the N-terminus and C-Cap region can be RI regions, retaining the hydrophobic core with L-amino acids.

For example, in using mirror-image phage display to screen for HRC variants, a first D-peptide can be synthesized from a HRN coronavirus peptide, or first L-peptide. The first L-peptide can be a naturally occurring L-peptide or can be a chimera of a peptide. The methods can further comprise screening for a HRC peptide, or second L-peptide, that specifically binds to the first D-peptide; then, a second D-peptide that is the mirror image of the second L-peptide can be synthesized. In one aspect of the D-peptide screening methods described herein, an N-trimer target can first be synthesized with D-amino acids, creating the mirror image of the natural L-N-trimer target. The D-N-trimer target can be used in standard peptide-based screens such as phage display, ribosome display, and/or CIS display to identify L-peptides that bind to the D-N-trimer. The identified L-peptides can then be synthesized with D-amino acids. By the law of symmetry, the resulting D-peptides bind the natural L-N-trimer and will thus target the N-trimer region of the coronavirus HRN intermediate, thereby inhibiting infection. This screening method is also described in Schumacher, et al., Identification of D-peptide ligands through mirror-image phage display, Science, 1996 Mar. 29; 271 (5257): 1854-7, which is hereby incorporated in its entirety by this reference.

The hotspot residues of the HRC peptide can be identified by crystal structure or NMR solution structure of the HRC peptide. For example, 1, 2, 3, 4, 5, 6, 7, 8, 9, 10 or more amino acids selected from 1168D, 1169I, 1172I, 1173N, 1176V, 1177V, 1179I, 1182E, 1189V, 1193L, 1194N, 1197L, 1198I, 1200L, 1202E and/or 1203L, such as one or more amino acids selected from 1177V, 1178N, 1181K, 1189V, 1198I, 1202E and/or 1203L of SEQ ID NO. 1 can be designated hotspot residues.

In some embodiments, the Peptide comprises a cell targeting peptide and at least one HRC peptide. In some embodiments, the targeting peptide is selected from but not limited to a receptor binding domain targeting peptide, an ACE2 targeting peptide. In some embodiments, the targeting peptide is an ACE2 targeting peptide.

In some embodiments where the peptide conjugate comprises only one Peptide, the peptide is HRC peptide derived from a coronavirus spike protein. In some embodiments where the peptide conjugate comprises 2 or more Peptides, each peptide can be a HRC peptide derived from a coronavirus spike protein. In some embodiments where the peptide conjugate comprises 2 or more Peptides, the Peptide can be selected from a HRC peptide derived from a coronavirus spike protein and/or a targeting peptide, provided that there is at least one HRC peptide derived from a coronavirus spike protein. Preferably, only one of the Peptides is a targeting peptide and the other peptide(s) are a HRC peptide derived from a coronavirus spike protein.

In some embodiments, the peptide conjugate of the invention is synthesized using click chemistry, and the Peptide comprises modified peptides such as modified HRC peptides or modified ACE2 targeting peptides, for example, proteins comprising a C-terminal or an N-terminal click chemistry handle. Such Peptides can then be covalently conjugated to B comprising a moiety that can react with the click chemistry handle of the Peptides. The term “chemistry handle” is defined as herein.

Paramyxoviruses infect human cells through the coordinated action of two surface glycoproteins: the attachment protein (H, HN, or G, depending on the virus) and the fusion (F) protein, a class I viral fusion protein embedded in the viral envelope. Unlike coronaviruses, where the spike protein performs both attachment and fusion functions, paramyxoviruses separate these roles. Depending on the type of virus type, the attachment protein binds to host cell receptors such as CD46, SLAM (CD150), or nectin-4. Upon receptor engagement, conformational signals are transmitted to the fusion protein, triggering a dramatic structural rearrangement that mediates membrane fusion between the viral envelope and host cell membranc.

The F protein is synthesized as an inactive precursor (F₀) and requires proteolytic cleavage into F₁ and F₂ subunits to become fusion-competent. The active F protein forms homotrimers that protrude from the viral surface and contains the essential elements for membrane fusion, including the fusion peptide (FP), heptad repeat 1 (HR1), and heptad repeat 2 (HR2) domains. During fusion, the HR1 and HR2 regions fold into a stable six-helix bundle, bringing the viral and host membranes into close proximity to facilitate fusion and viral entry. The F protein undergoes extensive conformational change similar to the class I fusion mechanism seen in other enveloped viruses.

Each peptide of the invention is independently a short amino acid sequence, including therapeutic peptides for paramyxovirus or peptide inhibitors that block paramyxovirus fusion, particularly by targeting the fusion protein. In some embodiments, each Peptide independently has a length ranging from 5 to 100 amino acid residues, from 6 to 80, from 8 to 60, or from 10 to 50 amino acid residues. In preferred embodiments, each Peptide independently has a length of 12-40 or more specifically 18-39 amino acid residues.

In some embodiments, each peptide is independently derived from a paramyxovirus fusion protein and/or target protein, preferably from the C-terminal heptad repeat (HR2 or HRC) region of the F protein. The HR2 region plays a key role in forming the six-helix bundle that drives membrane fusion. HR2-derived peptides mimic this region and can bind to the HR1 (or HRN) region of the F protein, thereby preventing bundle formation and inhibiting viral fusion and entry. In some embodiments, an HR2 peptide is a wild-type sequence derived from a paramyxovirus fusion protein, while in other embodiments, the HR2 peptide is a mutant or variant sequence that includes naturally occurring mutations or laboratory-optimized modifications.

In some embodiments, each peptide is independently an HR2-derived fusion inhibitor from a paramyxovirus F protein and/or a targeting peptide, provided that there is at least one HR2-derived fusion inhibitory peptide. While preferred embodiments utilize native or wild-type peptides, non-natural or engineered peptides may also be used. For example, amino acids associated with fusion-competent or fusion-incompetent variants of one strain may be combined with conserved sequences from other strains to generate broad-spectrum or more potent fusion inhibitors. The conserved nature of the fusion protein's HR1/HR2 regions across paramyxoviruses makes them attractive targets for developing pan-paramyxovirus inhibitors.

Preferred wild-type HR2 peptides comprise sequences and binding fragments thereof derived from the fusion protein of viruses such as measles virus, mumps virus, human parainfluenza virus 3 (HPIV3), or Nipah virus, among others.

In some embodiments, the peptide has at least about 70%, about 80%, about 90%, about 95, about 97%, or about 99% identity to a wild type HPIV3 HRC peptide:

(SEQ ID NO. 8)
VALDPIDISIELNKAKSDLEESKEWIRRSNQKLDSI.

In some embodiments, the peptide has at least about 70%, about 80%, about 90%, about 95, about 97%, or about 99% identity to a modified HPIV3 HRC peptide:

(SEQ ID NO. 9)
VALDPIDISIVLNKIKSDLEESKEWIRRSNKILDSI.

In some embodiments, the peptide has at least about 70%, about 80%, about 90%, about 95, about 97%, or about 99% identity to a modified HPIV3 HRC peptide:

(SEQ ID NO. 10)
VALDPIDISIVLNKIKSQLEESKWEIRRSNKILDSI.

In some embodiments, the peptide has at least about 70%, about 80%, about 90%, about 95, about 97%, or about 99% identity to a modified HPIV3 HRC peptide:

(SEQ ID NO. 11)
VALDPIDFSIVLNKIKSQLEESKWEIRRSNKILDSI.

In some embodiments, the peptide has at least about 70%, about 80%, about 90%, about 95, about 97%, or about 99% identity to a Measles HRC peptide:

(SEQ ID NO. 12)
ISLERLDVGTNLGNAIAKLEDAKELLESSDQILRSM.

In some embodiments, the peptide has at least about 70%, about 80%, about 90%, about 95, about 97%, or about 99% identity to a Nipah HRC peptide:

(SEQ ID NO. 13)
VFTDKVDISSQISSMNQSLQQSKDYIKEAQRLLDTV.

In some cases, the peptides of the invention are designed as pan-inhibitors, meaning they are capable of inhibiting multiple related targets within a viral family or class of proteins. Such peptides may block a broad range of viruses or biological pathways, thereby providing robust and versatile therapeutic potential.

In some cases, the peptides are engineered as selective inhibitors, targeting a specific virus or protein with high specificity. These selective inhibitors are designed to minimize off-target effects while maximizing potency against the intended pathogen.

In some cases, the peptides function as pan-paramyxovirus inhibitors, capable of inhibiting a wide array of paramyxoviruses, including measles virus, mumps virus, parainfluenza (HPIV3) viruses, and Nipah virus. These peptides may disrupt conserved regions of viral fusion machinery, providing broad-spectrum antiviral activity across the paramyxovirus family.

In some cases, the peptides are developed as measles-selective inhibitors, designed specifically to block fusion or entry of the measles virus by targeting unique features of the measles fusion protein.

In some cases, the peptides are tailored as mumps virus-selective inhibitors, focusing on the inhibition of mumps virus fusion or replication processes without substantially affecting other related viruses.

In some cases, the peptides are designed as parainfluenza virus-selective inhibitors (HPIV3-selective inhibitors), specifically interfering with the entry or fusion machinery of parainfluenza viruses, thereby preventing infection while minimizing impacts on other paramyxoviruses.

In some cases, the peptides are engineered as Nipah virus-selective inhibitors, targeting critical regions of the Nipah virus fusion protein to block its ability to mediate membrane fusion and viral entry.

In some cases, the peptides are designed as pan-coronavirus inhibitors, capable of inhibiting multiple coronaviruses, such as SARS-COV, MERS-COV, and SARS-COV-2, by targeting conserved regions of the viral spike protein or essential viral enzymes. Such peptides may provide broad-spectrum protection against known and emerging coronaviruses.

In some cases, the peptides are optimized as SARS-COV-2-selective inhibitors, designed to interfere with the viral spike protein's ability to bind to host receptors or mediate membrane fusion, thereby preventing infection by SARS-COV-2 specifically.

In some cases, the peptides are developed as MERS-COV-selective inhibitors, targeting unique structural features of the MERS coronavirus spike protein to inhibit viral entry and prevent infection.

### Linker

Each Linker is independently optional. Each Linker is independently a bivalent moiety that covalently binds to a Peptide and to B. Each Linker independently comprises ester, amide, disulfide, thiol, peptide, or polymeric moiety (such as polyethylene glycol (PEG)).

Each Linker can independently have 1, 2, 3, 4, 5 or more subunits or segments. In some embodiments, each Linker independently comprises a subunit with one or more amino acids. The amino acids may be naturally occurring or synthetic. Thus, the Linker may comprise (Gly)ₙ₊₁, (GlySerGly)ₙ or (Gly-Pro)ₙ where n is 1 or greater, for example, 1 to 12, 1 to 6 or 1 to 4. GlySerGly is one example of a sequence of amino acids which may form the Linker or part of the Linker.

In some embodiments, the Linker may comprise a non-amino acid subunit. In some embodiments, examples of the non-amino acid subunit of the linker are —(OCH₂CH₂)ₘ— where m is from 1 to 15, for example 2 to 10, 2 to 6 or 4. Introduction of a (poly)ethyleneglycol group assists solubility in aqueous media. In some embodiments, examples of the non-amino acid portion of the linker are —CH₂C(O)— and —CH₂C(O)NHCH₂CH₂(OCH₂CH₂)₄C(O)—.

For example, it can be advantageous to use a Linker with 3 subunits. A first optional subunit which comprises a flexible peptide, such as -(G)ₘ- or -(GS)ₘG-, where m is an integer of 1, 2, 3, 4, 5 or more, such as 2. A second subunit can be a residue of a chemical reaction (such as an automated flow chemistry reaction), such as a peptide bond, ester, or ether involving the N-terminus, C-terminus or side chain of the Peptide or first subunit. The residue can be non-cleavable, such as that formed with carbodiimide or sulfhydryl maleimide. A third optional subunit can be a hydrophilic spacer, a PEG spacer, such as polyethyleneglycol, polyethyleneamine, polyacetal polymer, poly(l-hydroxymethylethylene hydroxymethyl-formal) (PHF) or a carbohydrate. The hydrophilic spacers can generally be polymeric and comprise 1, 2, 3, 4, 5, 6, 7, 8, 9, 10 or more monomers. Polyethyleneglycol with 4 monomers (PEG4) is satisfactory. When the Peptide that a linker is connected to is a HRC peptide, the length of the hydrophilic spacer can correspond to the span of the protein gap to facilitate the orientation of the HRC peptide to bind the HRN domain.

Each Linker can be independently designed to modify the activities of the peptide conjugate, such as flexible linkers to increase flexibility or rigid linkers to maintain a fixed distance. Chen et al. “Fusion Protein Linkers: Property, Design and Functionality”, Adv Drug Deliv Rev. 2013 65(10):1357-69. Preferably, when the Peptide is a HRC peptide, the Linker is selected from a flexible linker to facilitate the orientation of the HRC peptide to bind the HRN domain. In other preferred embodiments, the Linker is a rigid linker to facilitate the manufacturing of the peptide sequences.

Suitable peptide linkers include polypeptides of between about 1 amino acid and about 40 amino acids in length, or between about 3 amino acids and about 25 amino acids in length. Peptide linkers with a degree of flexibility can be used. The use of small amino acids, such as glycine (G or Gly), serine (S or Ser), and alanine (A or Ala), are of use in creating a flexible peptide. A variety of different linkers are commercially available and are considered suitable for use.

Examples of each Linker include glycine polymers (G) n, glycine-serine polymers (including, for example, (GS)ₙ, (GSGGS)ₙ, (GGSGGS)ₙ, (GGGS)ₙ, where n is an integer of at least one), glycine-alanine (G-A) polymers, alanine-serine (A-S) polymers. Exemplary Linker can comprise amino acid sequences including, but not limited to, GGSG, GGSGG, GSGSG, GSGGG, GGGSG, GSSSG, and the like.

### B Moiety

B is a multimeric core which provides a framework that covalently links the one or more Peptide-Linker moieties to the hydrophobic moiety.

In some embodiments, the peptide conjugate of the invention is synthesized using click chemistry. Click chemistry handles are chemical moieties that provide a reactive group that can partake in a click chemistry reaction. Click chemistry reactions and suitable chemical groups for click chemistry reactions are well known to those of skill in the art, and include, but are not limited to terminal alkynes, azides, strained alkynes, dienes, dieneophiles, alkoxyamines, carbonyls, phosphines, hydrazides, thiols, and alkenes. For example, in some embodiments, an azide and an alkyne are used in a click chemistry reaction.

In some embodiments where copper-catalyzed azide-alkyne cycloaddition (CuAAC) is the click-chemistry employed for functionalizing materials as disclosed herein, the “click-chemistry compatible” compounds include a terminal alkyne and/or terminal azide functional group.

An exemplary click-chemistry reaction is CuAAC, although skilled artisans will appreciate that other click-chemistry compatible reactions that would be appreciated as equivalent to CuAAC may be employed without departing from the scope of the inventive concepts described herein. For instance, in various embodiments click-chemistry compatible reactions may include CuAAC, strain-promoted azide-alkyne cycloaddition (SPAAC), strain-promoted alkyne-nitrone cycloaddition (SPANC), strained alkene reactions such as alkene-azide cycloaddition, etc. Click-chemistry compatible reactions may also be considered to include alkene-tetrazine inverse-demand Diers-Alder reactions, alkene-tetrazole photoclick reactions, Michael additions of thiols, nucleophilic substitution of thiols with amines, and certain Diels-Alder reactions, etc. such as disclosed by Becer, et al. “Click chemistry beyond metal-catalyzed cycloaddition.” Angew. Chem. Int. Ed. 2009, 48: p. 4900-4908, and equivalents thereof as would be understood by a person having ordinary skill in the art upon reading the present disclosures.

Accordingly, click-chemistry compatible groups, compounds, etc. should be understood to include one or more suitable chemical moieties conveying capability to participate in any combination of the foregoing exemplary click chemistries, in various embodiments.

In some embodiments, B comprises a moiety that is derived from a compound comprising a thiol group that facilitates the click-chemistry reactions described herein. The compound for example is cysteine. In some embodiments, B comprises one or more cysteine residue, one or more X, and optionally Y, and/or optionally Z, wherein X, Y and Z are defined herein.

The one or more cysteine residue, one or more X, optional Y, and optional Z can be in any order, wherein the component of B listed first is bound to the Peptide-Linker and the component listed last is bound to the Hydrophobic Moiety. For example, wherein B comprises, in order, one or more cysteine residue, one or more X, and Z, the Peptide-Linker is bound to the one or more cysteine and Z is bound to the Hydrophobic Moiety. In some embodiments, B comprises, in order, one or more cysteine and one or more X.

In some embodiments, B comprises cysteine and X.

In some embodiments, B comprises one or more cysteine, one or more X and Z. In some embodiments, B comprises Z, one or more cysteine, and one or more X.

In some embodiments, B comprises Y, one or more cysteine, and one or more X. In some embodiments, B comprises Y, one or more cysteine, one or more X, and Z.

In some embodiments, the one or more cysteine binds the one or more Peptide-Linker moieties to the other components of B. In some embodiments, the one or more cysteine is attached to the one or more X via a thioether bond. In some embodiments, the one or more cysteines have the following structure:

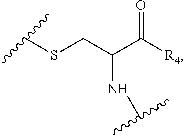

wherein R₄ is OH or NH₂, the —S-bond is covalently linked with X, the —NH-bond is covalently linked with a Peptide-Linker directly or indirectly via one or more Y.

In some embodiments, X comprises one or more sulfur aryl linkage, nitrogen aryl linkage or other linkages such as triazoles, amides, sulfur-sp3 carbon bonds, or a hydrophilic linker. In some embodiments, hydrophilic linker is selected from such as polyethyleneglycol (PEG), polyethyleneimine, polyacetal polymer, poly(l-hydroxymethylethylene hydroxymethyl-formal) (PHF) or a carbohydrate. In some embodiments, the hydrophilic linker can be polymeric and comprise 1, 2, 3, 4, 5, 6, 7, 8, 9, 10 or more monomers. In some embodiments, the hydrophilic linker is polyethyleneglycol (PEG). In some embodiments, the hydrophilic linker is polyethyleneglycol with 4 monomers (PEG4). In one embodiment X comprises one or more sulfur aryl linkage. In one embodiment X comprises one or more nitrogen aryl linkage. In one embodiment, X comprises one or more sulfur-sp3 carbon bonds.

In some embodiments, X is represented by -R_{A}-X_{I}-R_{B}-, wherein the left of R_{A} is covalently linked to cysteine via a thioether bond and the right of R_{B} is covalently linked to Hydrophobic Moiety directly or indirectly via one or more Z; R_{A} is selected from a substituted or unsubstituted alkyl group, a substituted or unsubstituted aryl group, a substituted or unsubstituted heteroaryl group, a substituted or unsubstituted cycloalkyl group, or a substituted or unsubstituted cycloheteroalkyl group, such as a —(CH₂)₁₋₆—, a substituted or unsubstituted phenyl, a substituted or unsubstituted 5- to 8-membered cycloalkyl, a substituted or unsubstituted 5- or 6-membered cycloheteroalkyl; X₁ is —C(O)O—, —OC(O)—, —SC(O)—, —C(O) NH—, —NHC(O)—, —(O)CS—, —SONH—, —NHCONH—, —S(O)—, or —S(O)₂—; R_{B} is a substituted or unsubstituted alkyl group, a —(CH₂O)₁₋₈—, —(CH₂CH₂O)₁₋₈—, —(OCH₂)₁₋₈-, or —(OCH₂CH₂)₁₋₈—.

In some embodiments, the one or more X is attached via a thioether bond directly with the hydrophobic moiety. In some embodiments, the one or more X is attached to Z, when present, which is then attached to the hydrophobic moiety.

In some embodiments, B further comprises Y. In some embodiments, Y, when present, the one or more Peptide-Linker moieties to the one or more cysteine, which then binds to the other components of B.

In some embodiments, Y comprises one or more amino acids. The amino acids may be naturally occurring or synthetic. Y may comprise 1 or more amino acids, for example, 1 to 12, 1 to 6 or 1 to 4. The one or more amino acids can be added to the linker in stepwise fashion. For example, a first amino acid is added to the cysteine of B and then, prior to the addition of a second amino acid, a Peptide-Linker is attached to the first amino acid. After attachment of the Peptide-Linker to the first amino acid, a subsequent amino acid is attached to the previous amino acid and allows for the attachment of a further Peptide-Linker and so on. In some embodiments, the amino acid of the linker is one or more diamino acids, such as lysine, arginine, ornithine, diaminopimelic acid (DAP). In some embodiments, the amino acid of the linker is one or more lysinc.

In some embodiments, Z, when present, comes between X and the hydrophobic moiety and binds B to the hydrophobic moiety.

In some embodiments, Z, when present, comprises a moiety having a structure according to formula (I):

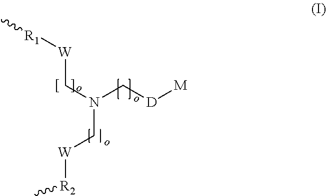

wherein each of R₁ and R₂ is independently selected from the group consisting of:
(i) absent
(ii) a structure according to formula (II):

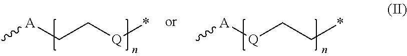

and
(iii) a structure according to formula (III):

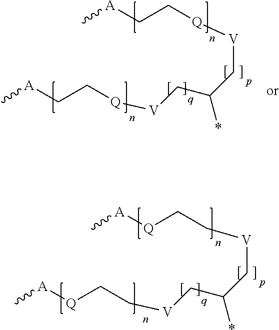

### (III);

wherein [symbol] represents a bond covalently linked to X;
W is in each instance independently selected from —C(O)O—, —OC(O)—, —O—, C(O)—, —(CH₂)ₘ—, and —NHC(O)—, most preferably W is —C(O) NH—;
V is in each instance independently selected from —(CH₂)ₘ—, —(CH₂)ₘ—C(O)—, —C(O)—(CH₂)ₘ—, —(CH₂)ₘ—C(O)—O—, —O—C(O)—(CH₂)ₘ—, —(CH₂)ₘ—C(O)—O—, —O—C(O)—(CH₂)ₘ—, —C(O)O—, and —OC(O)—; most preferably
V is —CH₂CH₂—C(O) NH—;
D is in each instance either —O— or —S—;
A is in each instance independently selected from —C(O)CH₂—, —CH₂C(O)—, —CH₂—, —C(O)—, —CH₂C(O)—, and —C(O)CH₂—; most preferably A is —CH₂—;
Q is in each instance independently selected from —CH₂—, —O—, —CH₂O—, and —OCH₂—; most preferably Z is —O—;
R3 is in each case independently selected from any of said polypeptides, which may be the same or different;
m is in each instance independently selected from an integer of between 0 and 5, i.e., 0, 1, 2, 3, 4, or 5; preferably between 0 and 3, preferably m is the same in each instance;
n is in each instance independently selected from an integer of between 0 and 40, i.e., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40; preferably between 3 and 10, preferably n is the same in each instance;
is in each case independently selected from an integer of between 0 and 5, i.e., 0, 1, 2, 3, 4, or 5; preferably 1 or 2, preferably o is the same in each instance;
p is in each instance independently selected from an integer of between 0 and 5, i.e., 0, 1, 2, 3, 4, or 5; preferably between 0 and 3, preferably p is the same in each instance;
q is in each instance independently selected from an integer of between 0 and 5, i.e., 0, 1, 2, 3, 4, or 5; preferably between 0 and 3; preferably q is the same in each instance and/or preferably q<p;
M represents the Hydrophobic Moiety; and
wherein * marks, where the structures (II-III) are linked to structure (I).

In some embodiments, Z, when present, comprises a moiety having a structure according to formula (IV):

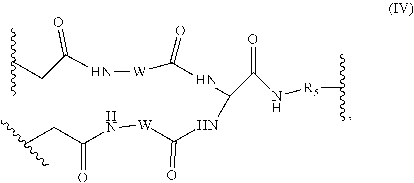

wherein R₅ is selected from hydrophilic linker such as polyethyleneglycol (PEG), polyethyleneimine, polyacetal polymer, poly(l-hydroxymethylethylene hydroxymethyl-formal) (PHF) or a carbohydrate; and
W is in each instance independently selected from direct bond, hydrophilic linker is selected from such as polyethyleneglycol (PEG), polyethyleneimine, polyacetal polymer, poly(l-hydroxymethylethylene hydroxymethyl-formal) (PHF) or a carbohydrate.

In some embodiments, the hydrophilic linker of R₅ can be polymeric and comprise 1, 2, 3, 4, 5, 6, 7, 8, 9, 10 or more monomers. In some embodiments, the hydrophilic linker of R₅ is polyethyleneglycol (PEG). In some embodiments, the hydrophilic linker of R₅ is polyethyleneglycol with 4 monomers (PEG4).

In some embodiments, the hydrophilic linker of W can be at each instance independently polymeric and comprise 1, 2, 3, 4, 5, 6, 7, 8, 9, 10 or more monomers. In some embodiments, the hydrophilic linker is polyethyleneglycol (PEG). In some embodiments, the hydrophilic linker of W can be at each instance independently polyethyleneglycol with 4 monomers (PEG4).

In some embodiments, the peptide conjugates are synthesized using automated flow chemistry, preferably using an automated flow peptide synthesis (AFPS) system. An automated flow peptide synthesis (AFPS), as disclosed in U.S. Pat. No. 10,683,325 B2 is a solid phase peptide synthesis system equipped with feedback control and can afford a high degree of control over individual coupling reactions for making peptides and/or minimize side reactions.

In any embodiments where the peptide-conjugates are synthesized using automated flow chemistry, B is a multivalent moiety that is designed to allow the rapid synthesis of the peptide conjugates via automated flow chemistry and can be tailored to covalently link the Peptide, the Hydrophobic Moiety, and other optional modules to improve anti-viral activities. B preferably comprises at least one diaminoaliphatic acids (such as diamino acids, lysine, arginine, ornithine, diaminopimelic acid (DAP)), optionally spacers, and optionally function groups such as amides, esters, and ethers. In some embodiments, B comprises 1, 2, 3, 4, 5, 6 or more diamino acids each independently selected from lysine, arginine, ornithine, and DAP. In some embodiments, B comprises 1, 2, 3, 4, 5, 6, or more lysines.

In some embodiments, B comprises one diamino acid selected from lysine, arginine, ornithine, and DAP; preferably one lysine. In some embodiments, B comprises two diamino acids each independently selected from lysine, arginine, ornithine, and DAP; preferably two lysines. In some embodiments, B comprises three diamino acids each independently selected from lysine, arginine, ornithine, and DAP; preferably three lysines. In some embodiments, B comprises four diamino acids each independently selected from lysine, arginine, ornithine, and DAP; preferably four lysines. In some embodiments, B comprises five diamino acids each independently selected from lysine, arginine, ornithine, and DAP; preferably five lysines. In some embodiments, B comprises six diamino acids each independently selected from lysine, arginine, ornithine, and DAP; preferably six lysines. When two or more diamino acids are present in B, the two or more diamino acids can be covalently linked to each other via amide bonds or via amino acid linkers such as GS linkers or polymeric linkers such as PEG linkers. In one preferable embodiment, B comprises two lysines covalently linked to each other via an amide bond. In additional preferable embodiments, B comprises one lysine. In yet additional embodiments, B comprises three lysines, covalently linked to each other via two amide bonds. In another embodiment, B comprises four lysines, covalently linked to each other via three amide bonds.

In some embodiments, when the diamino acid is lysine, B is represented by the Formula (B1):

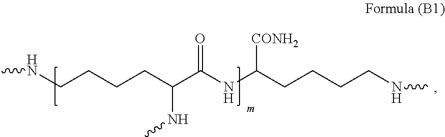

wherein [symbol] represents covalent bonds linking to the moieties of the compound that comprise one or more fusion peptide inhibitors, a membrane anchoring moiety, optionally one or more spike binding peptide, and optionally one or more targeting peptide; m is an integer that can be 0, 1, 2, 3, 4, or more. Preferably, [symbol] represents a covalent bond to —CO— group. Preferably, m is 0, 1, or 2.

In some embodiments, the peptide conjugate is represented by Formula (V):

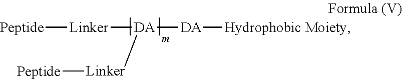

wherein Peptide, Linker, and Hydrophobic Moiety are as defined above, including all and preferable embodiments; m is 0, 1, 2, 3, 4, preferably, m is 0, 1, and 2; and each DA is independently selected from a diaminoaliphatic acid, preferably a diamino acid. In some embodiments, each Linker can be independently absent.

In some embodiments, each DA is independently selected from lysine, arginine, ornithine, and DAP. Preferably, each DA is lysine.

In some embodiments, B comprises one or more lysines, one or more spacers, and one or more additional function groups such as amides, esters, or ethers. The term “spacer”, as used herein, refers to a hydrophilic and biocompatible molecule or a chemical group that is inserted between two lysines, a lysine and a fusion peptide inhibitor, a lysine and a membrane anchoring moiety, a lysine and a spike binding peptide, or a lysine and a targeting peptide, to increase the distance between them. The spacer is used to avoid steric hindrance, reduce aggregation, improve solubility and the accessibility of the compound to the target. A common type of spacer used in the compounds is polyethylene glycol (PEG), which is a hydrophilic and biocompatible polymer that can increase the solubility and stability of the compounds in vivo. When a hydrophilic spacer PEG is present in B, a variety of PEG derivatives can be used for synthesizing the compound, such as, without limitation, amine-PEG-carboxyl acid, amine-PEG-maleimide, amine-PEG-biotin, amine-PEG-azido, azido-PEG-carboxyl acid, amine-PEG-NHS ester, maleimide-PEG-NHS ester, and biotin-PEG-NHS ester. An amine-PEG-carboxyl acid is preferably used for synthesizing the compound, preferably via automated flow chemistry, such as H₂N-PEG₁₋₄₀-COOH, H₂N-PEG₁₋₄₀-CH₂COOH, or H₂N-PEG₁₋₄₀-CH₂CH₂COOH. Other hydrophilic spacers can be used, such as polyethyleneamine, polyacetal polymer, poly(l-hydroxymethylethylene hydroxymethyl-formal) (PHF) or a carbohydrate. The length of the hydrophilic spacer can correspond to the span of the protein gap to facilitate the orientation of the HRC peptide to bind the HRN domain. B and the spacer, Linker and the spacer, or Peptide and the spacer can be joined to each other by the residue of a chemical reaction (such as an automated flow chemistry reaction).

Therefore, in some embodiments, B can be for example represented by the Formula (B2):

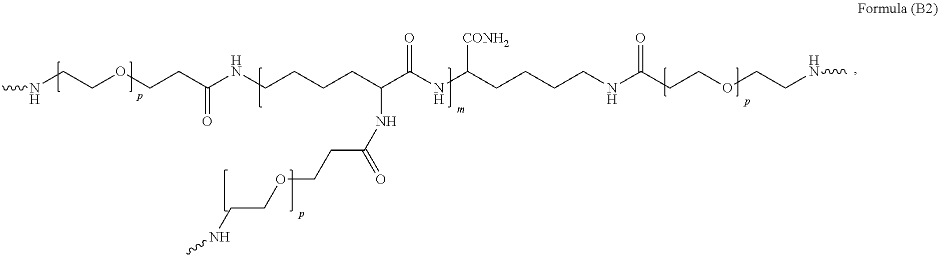

wherein [symbol] and m are as defined above, including all and preferable embodiments. p is an integer selected from 0 to 40.

When a spacer PEG is present in B, in some embodiments, the compound can be therefore represented by Formula (VI):

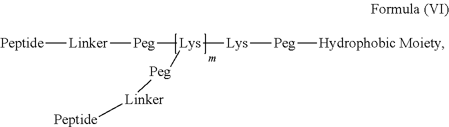

wherein Peptide, Linker, Hydrophobic Moiety are as defined above, including all and preferable embodiments; m is 0, 1, 2, 3, 4, preferably, m is 0, 1, and 2; and each Peg is independently selected from a PEG spacer.

With reference to Formula (VI), Hydrophobic Moiety is cholesterol and B is Formula (B2), resulting in the structure of the peptide conjugate as shown by Formula (VII):

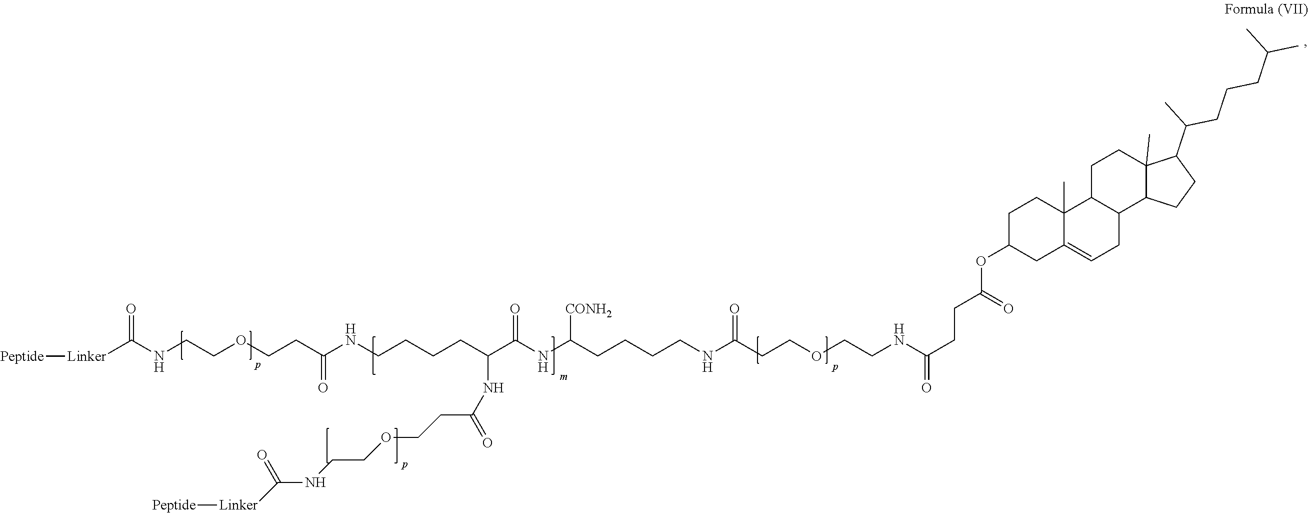

wherein Peptide, Linker, m, and p are as defined above, including all and preferable embodiments.

### Hydrophobic Moiety

Hydrophobic Moiety plays a role as an anchoring agent to anchor the peptide conjugate to cellular membrane so that the peptide can target the fusion process of the coronavirus and inhibit viral entry.

Hydrophobic moiety can be a lipid-based moiety including fats, waxes, steroids, cholesterol, fat-soluble vitamins, monoglycerides, diglycerides, triglycerides, phospholipids, sphingolipids, glycolipids, cationic or anionic lipids, derivatized lipids. Preferably Hydrophobic Moiety is a membrane integrating lipid including cholesterol, sphingolipid, sphingomyelin, glycolipid, glycerophospholipid (such as phosphatidylcholine, phosphatidylethanolamine and phosphatidylserine), ergosterol, 7-dihydrocholosterol and stigmasterol.

In some embodiments, the Hydrophobic Moiety can be cholesterol. Cholesterols can include, cholesterol, esters of cholesterol including cholesterol hemi-succinate, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate and lanosterol sulfate. In any embodiments wherein Hydrophobic Moiety is cholesterol, B is preferably linked directly or indirectly to a cholesterol hydroxyl group, such as 3-OH.

In some embodiments, the Hydrophobic Moiety can be a phospholipid. Phospholipids that can be used in this application include, without limitation, egg phosphatidylcholine (EPC), egg phosphatidylglycerol (EPG), egg phosphatidylinositol (EPI), egg phosphatidylserine (EPS), phosphatidylethanolamine (EPE), and phosphatidic acid (EPA); the soya counterparts, soy phosphatidylcholine (SPC); SPG, SPS, SPI, SPE, and SPA; the hydrogenated egg and soya counterparts (e.g., HEPC, HSPC), other phospholipids made up of ester linkages of fatty acids in the 2 and 3 of glycerol positions containing chains of 12 to 26 carbon atoms and different head groups in the I position of glycerol that include choline, glycerol, inositol, serine, ethanolamine, as well as the corresponding phosphatidic acids. The chains on these fatty acids can be saturated or unsaturated, and the phospholipid may be made up of fatty acids of different chain lengths and different degrees of unsaturation. In particular, the compositions of the formulations can include dipalmitoylphosphatidylcholine (DPPC), a major constituent of naturally-occurring lung surfactant. Other examples include dimyristoylphosphatidycholine (DMPC) and dimyristoylphosphatidylglycerol (DMPG) dipalmitoylphosphatideholine (DPPQ) and dipalmitoylphosphatidylglycerol (DPPG) distearoylphosphatidylcholine (DSPQ) and distearoylphosphatidylglycerol (DSPG), dioleylphosphatidyl-ethanolarnine (DOPE) and mixed phospholipids like palmitoylstearoylphosphatidyl-choline (PSPC) and palmitoylstearolphosphatidylglycerol (PSPG), and single acylated phospholipids like mono-oleoyl-phosphatidylethanolamine (MOPE).

In some embodiments, the Hydrophobic Moiety can be a sphingolipid, including sphingosine, sphingomyelins, cerebroside, sulfatides, globosides, gangliosides, galactocerebroside, glucocerebroside, GM2 ganglioside, GM1 ganglioside, and glycoplipids including ceramide trihexoside.

In some embodiments, the Hydrophobic Moiety can be a tocopherol. The tocopherols can include tocopherols, esters of tocopherols including tocopherol hemi-succinates, salts of tocopherols including tocopherol hydrogen sulfates and tocopherol sulfates.

In some preferable embodiments, the Hydrophobic Moiety is cholesterol.

B can be covalently connected to a convenient position on the Hydrophobic Moiety. In some embodiments, connection is via a hydroxy group of the Hydrophobic Moiety. For example, when the Hydrophobic Moiety is cholesterol, B can be connected to the cholesterol by a group —C(O)— or —C₁₋₄ alkylene C(O)—, such as —CH₂C(O)—.

Nonlimiting examples of the peptide conjugate structure are presented in Table 1:

TABLE 1 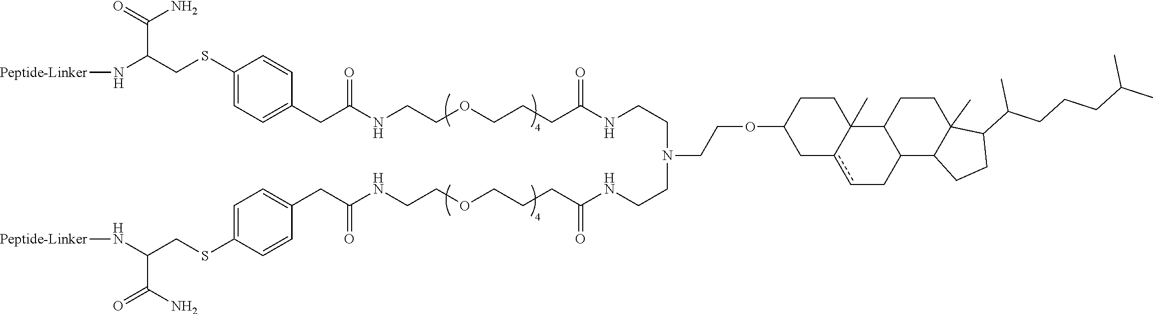
Peptide Conjugate 1 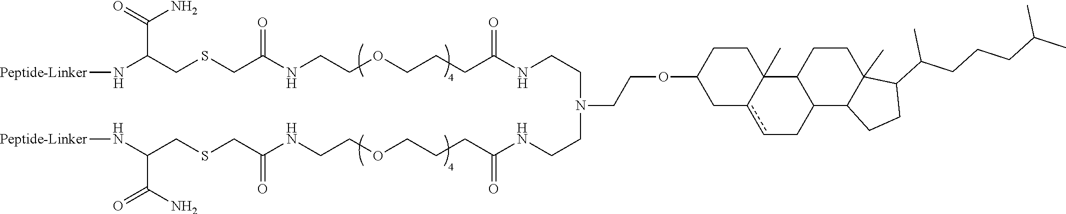
Peptide Conjugate 2 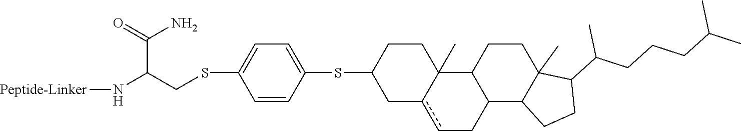
Peptide Conjugate 3 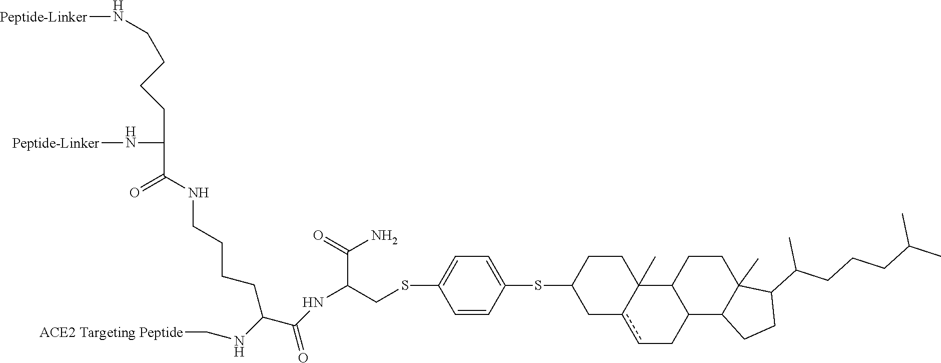
Peptide Conjugate 4 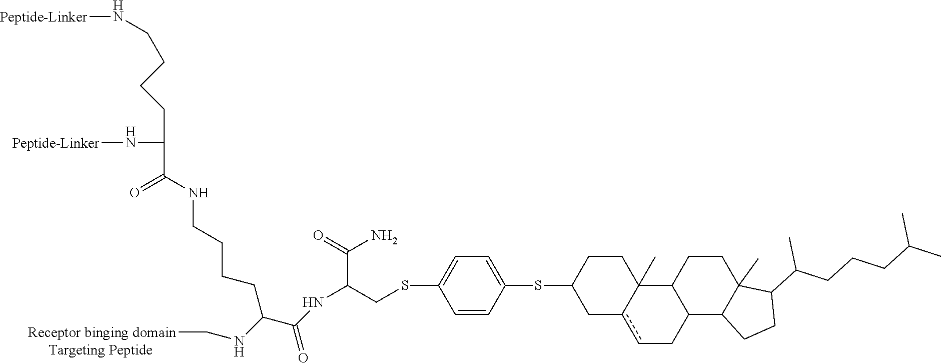
Peptide Conjugate 5

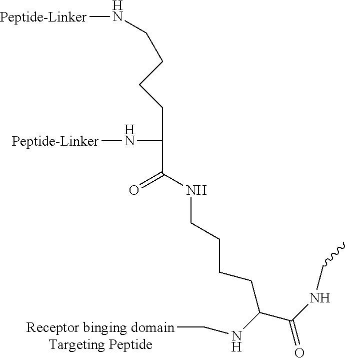

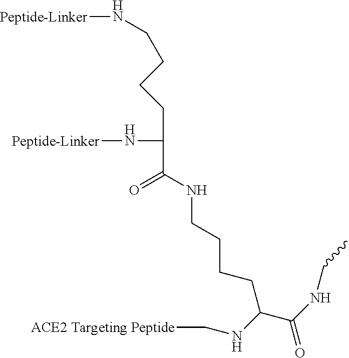

Peptide Conjugate 6 (left) and Peptide Conjugate 7 (right),
The NH group with [symbol] is bound to cholesterol via a linker. 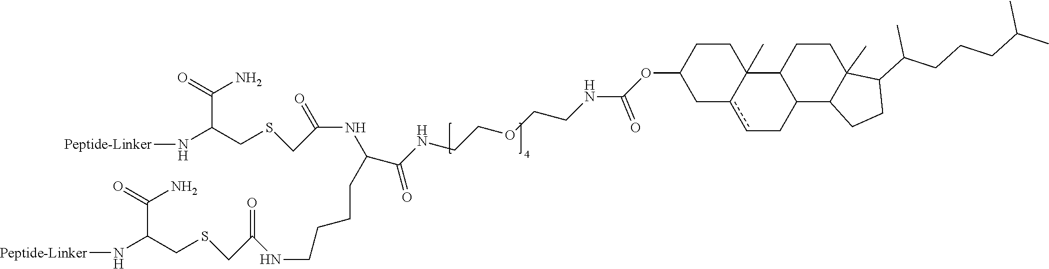
Peptide Conjugate DCOY101 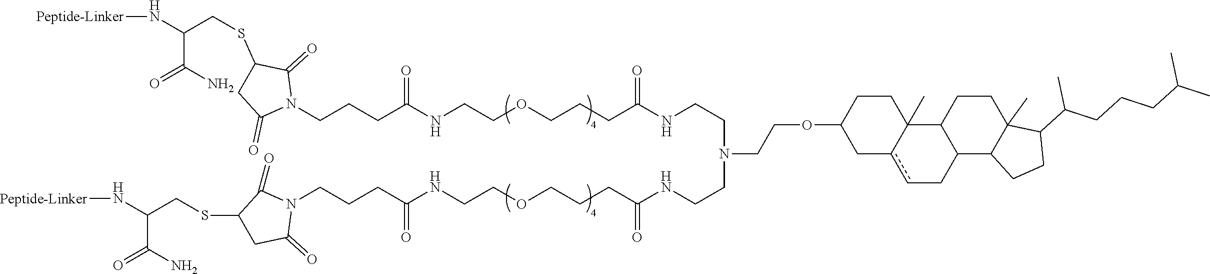
Peptide Conjugate DCOY102 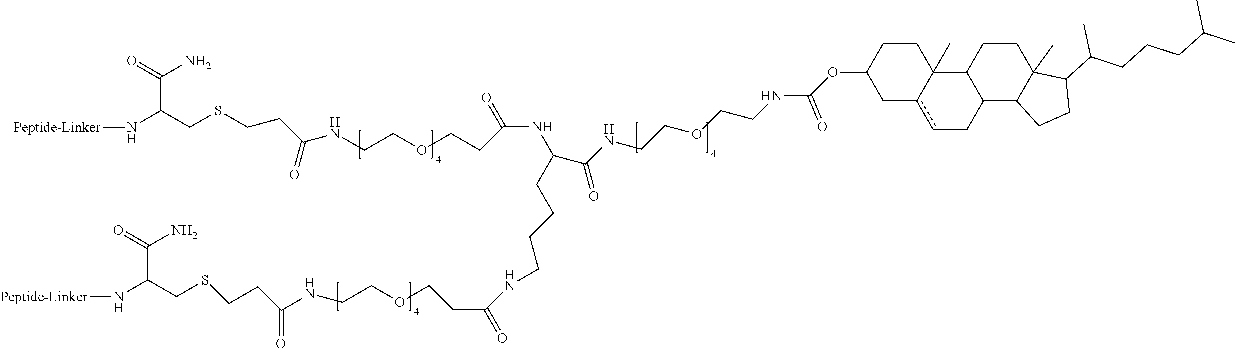
Peptide Conjugate DCOY103 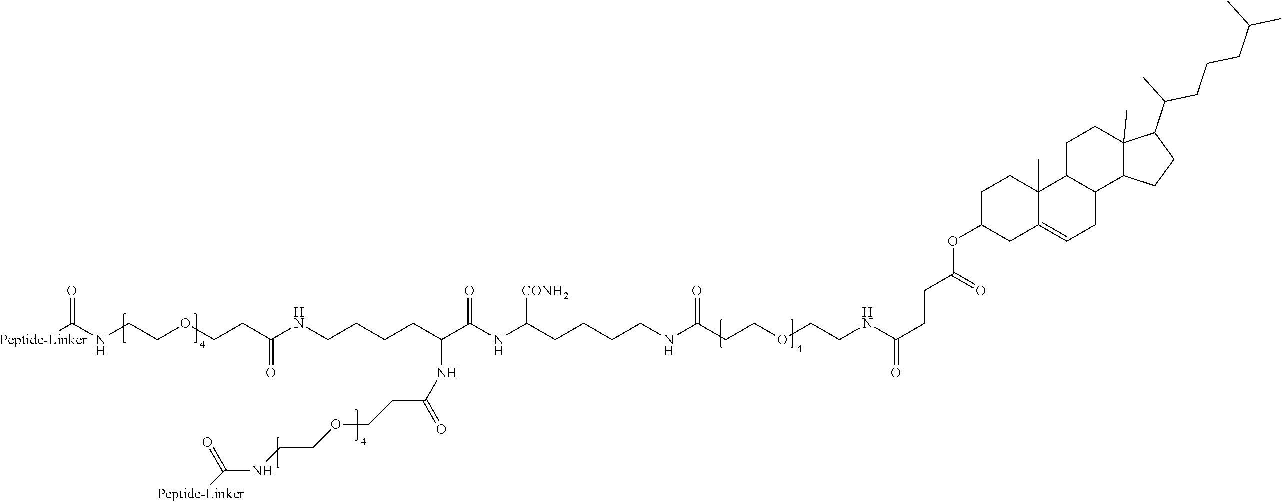
Peptide Conjugate DCOY104 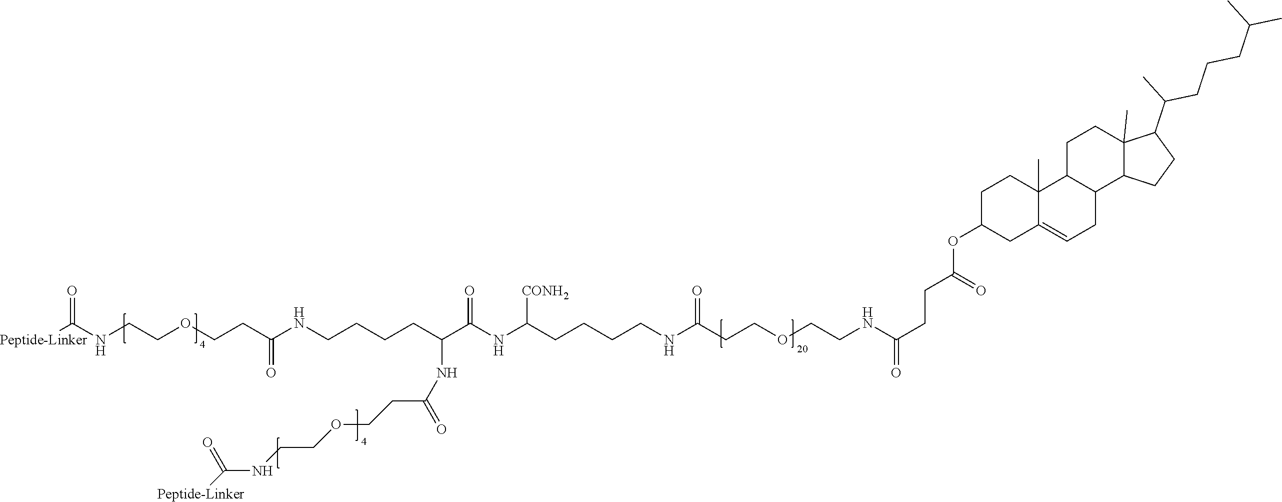
Peptide Conjugate 12.

Peptide-Linker for any one of the peptide conjugates (Peptide Conjugates 1-5 and DOY101-DCOY104) in Table 1 is SEQ ID NO. 5:

(SEQ ID NO. 5)
H2N-DISGINASVVNIQKEIDRLNEVAKNLNESLIDLQELGSGSG.

In additional embodiments, provided are also peptide conjugates that respectively comprise the B-Hydrophobic Moiety structures as presented in Table 1 with Peptide-Linker different from SEQ ID NO. 5. In those embodiments, with reference to the core structures in Table 1, each Peptide and each Linker are independently as defined above, including all additional and preferred embodiments.

Preferably, each Peptide is independently selected from SEQ ID NO. 1 and SEQ ID NO. 2:

Acₙ-DISGINASVVNIQKEIDRLNEVAKNLNESLIDLQEL (SEQ ID NO. 1), wherein n is 0 or 1.

(SEQ ID NO. 2)
dIdGdSdIdD NASVVNIQKEIDRLNEVAKNLNESLIDLQEL

In some embodiments, the Peptide-Linker is independently selected from SEQ ID NO. 3 and SEQ ID NO. 4:

(SEQ ID NO. 3)
DISGINASVVNIQKEIDRLNEVAKNLNESLIDLQELGSGSG
(SEQ ID NO. 4)
dIdGdSdIdD NASVVNIQKEIDRLNEVAKNLNESLIDLQELGSGSG.

In some cases, each Peptide is independently selected from SEQ ID NO. 8-13.

In some cases, each Peptide is independently selected from SEQ ID NO. 1-2 and 8-13.

Preferably, with reference to the core structures in Table 1, each Peptide-Linker is the same for the same peptide conjugate. Additionally, with reference to the core structures in Table 1, one Peptide-Linker is selected from a receptor binding domain targeting peptide, an ACE2 targeting peptide, and the other (Peptide-Linker) s are the same HRC peptide for the same peptide conjugate.

### Pharmaceutical Composition

The compositions of the invention comprise a peptide conjugate as described herein and a pharmaceutically acceptable carrier. For example, the composition can be administered systemically or locally. The composition can be administered for oral, intravenous, intramuscular, rectal, cutaneous, subcutaneous, topical, transdermal, sublingual, nasal, inhalation, or vaginal delivery, for example. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The compositions may be formulated according to conventional pharmaceutical practice (see, e.g., Remington: The Science and Practice of Pharmacy, 22^{nd} edition, 2013, ed. L. V. Allen, Pharmaceutical Press, Philadelphia, and Encyclopedia of Pharmaceutical Technology, 4.sup.th Edition, ed. J. Swarbrick, 2013, CRC Press, New York).

Peptide conjugates may be formulated in a variety of ways that are known in the art. For example, one or more peptide conjugates of the invention and any additional biologically active agent, if present, as defined herein may be formulated together or separately.

Each peptide conjugate of the invention, alone or in combination with one or more active agents as described herein, can be formulated for controlled release (e.g., sustained or measured) administration, as described in U.S. Patent Application Publication Nos. 2003/0152637 and 2005/0025765, each incorporated herein by reference. For example, a peptide conjugate of the invention, alone or in combination with one or more of the biologically active agents as described herein, can be incorporated into a capsule or tablet that is administered to the patient.

Controlled release formulations known in the art include specially coated pellets, polymer formulations or matrices for surgical insertion or as sustained release microparticles or nanoparticles, e.g., microspheres or microcapsules, for implantation, insertion, infusion or injection, wherein the slow release of the active medicament is brought about through sustained or controlled diffusion out of the matrix and/or selective breakdown of the coating of the preparation or selective breakdown of a polymer matrix. Other formulations or vehicles for controlled, sustained or immediate delivery of an agent to a preferred localized site in a patient include, e.g., lipid nanoparticles (LNP), suspensions, emulsions, gels, liposomes, and any other suitable art known delivery vehicle or formulation acceptable for subcutaneous or intramuscular administration.

Suitable biocompatible polymers can be utilized as the controlled release material. The polymeric material may comprise biocompatible, biodegradable polymers, and, in preferred embodiments, is preferably a copolymer of lactic and glycolic acid. Preferred controlled release materials which are useful in the formulations of the invention include the polyanhydrides, polyesters, co-polymers of lactic acid and glycolic acid (preferably wherein the weight ratio of lactic acid to glycolic acid is no more than 4:1 i.e., 80% or less lactic acid to 20% or more glycolic acid by weight) and polyorthoesters containing a catalyst or degradation enhancing peptide conjugate, for example, containing at least 1% by weight anhydride catalyst such as maleic anhydride. Examples of polyesters include polylactic acid, polyglycolic acid and polylactic acid-polyglycolic acid copolymers. Other useful polymers include protein polymers such as collagen, gelatin, fibrin and fibrinogen and polysaccharides such as hyaluronic acid.

In additional embodiments, the controlled release material, which in effect acts as a carrier for a peptide conjugate of the invention can further include a bioadhesive polymer such as pectins (polygalacturonic acid), mucopolysaccharides (hyaluronic acid, mucin) or non-toxic lectins or the polymer itself may be bioadhesive, e.g., polyanhydride or polysaccharides such as chitosan. In some embodiments where the biodegradable polymer comprises a gel, one such useful polymer is a thermally gelling polymer, e.g., polyethylene oxide, polypropylene oxide (PEO-PPO) block copolymer such as PLURONIC™ F127 from BASF Wyandotte.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, taste masking agents (such as hydroxypropyl methylcellulose, hydroxypropyl cellulose) and the like.

One or more peptide conjugates of the invention may be mixed together in a tablet, capsule, or other vehicle, or may be partitioned. In one example, a peptide conjugate of the invention is contained on the inside of the tablet, and the biologically active agent is on the outside of the tablet, such that a substantial portion of the biologically active agent is released prior to the release of the peptide conjugate of the invention.

Formulations for oral use may also be provided as chewable tablets, or as hard gelatin capsules wherein the active ingredient(s) are mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment. Formulations to the mouth may also be provided as a mouthwash, an oral spray, oral rinse solution, or oral ointment, or oral gel.

Dissolution or diffusion controlled release can be achieved by appropriate coating of a tablet, capsule, pellet, or granulate formulation of peptide conjugates, or by incorporating the peptide conjugate into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

Liquid forms in which the peptide conjugates and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Formulations suitable for parenteral administration (e.g., by injection), include aqueous or non-aqueous, isotonic, pyrogen-free, sterile liquids (e.g., solutions, suspensions), in which the peptide conjugate is dissolved, suspended, or otherwise provided (e.g., in a liposome or other microparticulate). Such liquids may additionally contain other pharmaceutically acceptable ingredients, such as anti-oxidants, buffers, preservatives, stabilizers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the peptide conjugate in the liquid is from about 1 ng/ml to about 10 ug/ml, for example from about 10 ng/ml to about 1 ug/ml. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

The composition of the invention can comprise a liquid vehicle which is suitable for nasal administration. The vehicle is preferably an aqueous solution or a mixed aqueous and organic solution (e.g., saline and propylene glycol). An aqueous solution includes a viscosity enhancing agent and, optionally, one or more additional excipients which, for example, improve formulation stability and/or comfort upon administration. More preferably, the vehicle is a mixed aqueous and organic solution (e.g., saline and propylene glycol).

A variety of viscosity enhancing agents are known in the art. Viscosity enhancing agents include hydrophilic polymers, such as polysaccharides, polysaccharide derivatives, proteins and synthetic polymers. Examples include, but are not limited to, acacia, tragacanth, alginic acid, carrageenan, locust bean gum, guar gum, gelatin, hyaluronic acid, polyacrylate, polyacrylate/alkylacrylate copolymers, polyvinyl alcohol, polyvinylpyrrolidone, starch, propylene glycol alginate, maltodextrin, and cellulose ether derivatives, such as methylcellulose, hydroxyethylcellulose, hydroxypropylmethylcellulose, hydroxypropylcellulose, and carboxymethylcellulose. Where possible, salt forms of any of the foregoing are preferred. Preferred viscosity enhancing agents include hyaluronic acid, including sodium hyaluronate; carboxymethylcellulose, including sodium carboxymethylcellulose and calcium carboxymethylcellulose; methylcellulose, hydroxyethylcellulose, hydroxypropylmethylcellulose, and hydroxypropylcellulose.

The composition optionally includes one or more additional excipients which, for example, increase the case of administration, the comfort of the subject, or the stability of the composition. Suitable additional excipients include, but are not limited to, tonicity modifiers, such as sodium chloride and dextrose; antioxidants, such as butylated hydroxyanisole; buffers, such as sodium bicarbonate, sodium citrate and sodium phosphate; preservatives, such as benzalkonium chloride, ethanol, propylene glycol, benzoyl alcohol, phenethyl alcohol, chlorobutanol or methylparaben; pH adjusters, such as hydrochloric acid, sulfuric acid and sodium hydroxide; surfactants, such as Polysorbate 80, Polysorbate 20, and polyoxyl 400 stearate; chelating agents, such as disodium EDTA; antioxidants; co-solvents, such as ethanol, PEG 400, and propylene glycol; penetration enhancers, such as oleic acid; and humectants, such as glycerin (see S. Thorat, Sch. J. App. Med. Sci. 2016, 4 (8D): 2976-2985; D. Marx et al., IntechOpen, DOI: 10.5772/59468. Available from: intechopen.com/books/drug-discovery-and-development-from-molecules-to-medicine/intranasal-drug-administration-an-attractive-delivery-route-for -some-drugs).

In one embodiment, the vehicle consists of sodium hyaluronate, aloe vera, allantoin, sodium chloride, sodium bicarbonate, glycerin, propylene glycol, propylene glycol, benzalkonium chloride and USP grade purified water. A suitable vehicle is sold by NEILMED™ under the tradename NASOGEL™.

The amount of active agent in the composition can vary, for example, from about 0.5% by weight to about 25% by weight.

The pH of the formulation is tolerable in the nasal cavity and preferably in the range of about 5.0 to about 8.0. Buffers that can be used in the formulation include, but are not limited to phosphate, TRIS, [tris(hydroxymethyl)methylamino] propanesulfonic acid, 2-(bis(2-hydroxyethyl)amino) acetic acid, and N-[tris(hydroxymethyl)methyl]glycine, and Alkaline Buffer (Seachem).

A pharmaceutical composition suitable for nasal or pulmonary administration comprising a water soluble solvent selected from the group consisting of propylene glycol, glycerin, polyethylene glycol, and combinations thereof. The composition can further comprise one or more of a polysaccharide gum, a non-ionic surfactant, and a preservative. An exemplary polysaccharide gum is sclerotium gum. Exemplary surfactants are poloxamers, including, but not limited to, poloxamer 188. The preservative can, for example, be benzalkonium chloride.

The composition can be a dry powder and delivered by a dry powder inhaler, suspended in a propellant or in an aqueous suspension or solution and delivered via a nebulizer.

For example, a solution or suspension of the active agent and a pulmonary excipient, such as lactose, can be spray dried to form particles having a fine particle fraction sufficient to deliver to the lungs or upper respiratory system. Alternatively, an aqueous solution or suspension can be sonicated, thereby aerosolizing the solution/suspension to a droplet size that can be inhaled, e.g., via a nebulizer.

Excipients include carbohydrates including monosaccharides, disaccharides and polysaccharides. For example, monosaccharides such as dextrose (anhydrous and monohydrate), galactose, mannitol, D-mannose, sorbitol, sorbose and the like; disaccharides such as lactose, maltose, sucrose, trehalose, and the like; trisaccharides such as raffinose and the like; and other carbohydrates such as starches (hydroxyethylstarch), cyclodextrins and maltodextrins. Other excipients suitable for use with the present invention, including amino acids, are known in the art such as those disclosed in WO 95/31479, WO 96/32096, and WO 96/32149. Mixtures of carbohydrates and amino acids are further held to be within the scope of the present invention. The inclusion of both inorganic (e.g., sodium chloride, etc.), organic acids and their salts (e.g., carboxylic acids and their salts such as sodium citrate, sodium ascorbate, magnesium gluconate, sodium gluconate, tromethamine hydrochloride, etc.) and buffers is also contemplated.

The compositions may be used in the form of dry powders or in the form of stabilized dispersions comprising a non-aqueous phase. Accordingly, the dispersions or powders of the present invention may be used in conjunction with metered dose inhalers (MDIs), dry powder inhalers (DPIs), atomizers, nebulizers or liquid dose instillation (LDI) techniques to provide for effective drug delivery. With respect to inhalation therapies, those skilled in the art will appreciate that the hollow and porous microparticles of the present invention are particularly useful in DPIs. Conventional DPIs comprise powdered formulations and devices where a predetermined dose of medicament, either alone or in a blend with lactose carrier particles, is delivered as an aerosol of dry powder for inhalation.

The medicament is formulated in a way that it readily disperses into discrete particles with a mass median aerodynamic diameters of the powders will characteristically range from about 0.5-10, preferably from about 0.5-5.0 microns MMAD.

As discussed above, the stabilized dispersions disclosed herein may also be administered to the nasal or pulmonary air passages of a patient via aerosolization, such as with a metered dose inhaler. MDIs are well known in the art and could easily be employed for administration of the claimed dispersions without undue experimentation. Breath activated MDIs, as well as those comprising other types of improvements which have been, or will be, developed are also compatible with the stabilized dispersions and present invention and, as such, are contemplated as being within the scope thereof. However, it should be emphasized that, in preferred embodiments, the stabilized dispersions may be administered with an MDI using a number of different routes including, but not limited to, topical, nasal, pulmonary or oral. Those skilled in the art will appreciate that, such routes are well known and that the dosing and administration procedures may be easily derived for the stabilized dispersions of the present invention.

Along with the aforementioned embodiments, the stabilized dispersions of the present invention may also be used in conjunction with nebulizers as disclosed in PCT WO 99/16420, the disclosure of which is hereby incorporated in its entirety by reference, in order to provide an aerosolized medicament that may be administered to the pulmonary air passages of a patient in need thereof. Nebulizers are well known in the art and could easily be employed for administration of the claimed dispersions without undue experimentation. Breath activated nebulizers, as well as those comprising other types of improvements which have been, or will be, developed are also compatible with the stabilized dispersions and present invention and are contemplated as being within the scope thereof.

Along with DPIs, MDIs and nebulizers, it will be appreciated that the stabilized dispersions of the present invention may be used in conjunction with liquid dose instillation or LDI techniques as disclosed in, for example, WO 99/16421 hereby incorporated in its entirety by reference. Liquid dose instillation involves the direct administration of a stabilized dispersion to the lung. In this regard, direct pulmonary administration of bioactive peptide conjugates is particularly effective in the treatment of disorders especially where poor vascular circulation of diseased portions of a lung reduces the effectiveness of intravenous drug delivery. With respect to LDI the stabilized dispersions are preferably used in conjunction with partial liquid ventilation or total liquid ventilation. Moreover, the present invention may further comprise introducing a therapeutically beneficial amount of a physiologically acceptable gas (such as nitric oxide or oxygen) into the pharmaceutical microdispersion prior to, during or following administration.

### Combination Therapies

The peptide conjugate or composition described herein can be co-administered with other active agents and therapies.

In some embodiments, the other active agent includes, but is not limited to, antibodies against a paramyxovirus such as HPIV-3. For example, the protective antibodies PI3-E12 against HPIV-3 are described in Boonyaratanakornkit et al., “Protective antibodies against human parainfluenza virus type 3 infection”, mAbs, Volume 13, 2021, Issue 1, https://doi.org/10.1080/19420862.2021.1912884.

In some embodiments, the other active agent includes, but is not limited to, antibodies against SARS-COV-2. Suitable antibodies are described in, for example, US 2022/0017604, US 2022/0017614; US 2021/0403550, US 2021/0395345, US 2021/0403537; US 2021/0388066, US 2021/0388065, US 2021/0347859, or US 2021/0309733, which are incorporated herein by reference. In some embodiments, the antibody is a monoclonal antibody such as casirivimab, imdevimab, bamlanivimab, or etesevimab. In some embodiments, the antibody is a monoclonal antibody therapy such as casirivimab plus imdevimab, bamlanivimab, or bamlanivimab plus etesevimab.

In some embodiments, the other active agent includes, but is not limited to, the measles, mumps, and rubella vaccine (MMR vaccine).

The active agents and compositions of the present invention are also intended for use with general care provided patients with viral infections, including parenteral fluids (including dextrose saline and Ringer's lactate) and nutrition, antibiotic (including metronidazole and cephalosporin antibiotics, such as ceftriaxone and cefuroxime) and/or antiviral prophylaxis, fever (e.g., acetaminophen) and pain medication, antiemetic (such as metoclopramide) and/or antidiarrheal agents, vitamin and mineral supplements (including Vitamin K and zinc sulfate), anti-inflammatory agents (such as ibuprofen), pain medications, and medications for other common diseases in the patient population, such as artemether, artesunate-lumefantrine combination therapy), quinolone antibiotics, such as ciprofloxacin, macrolide antibiotics, such as azithromycin, cephalosporin antibiotics, such as ceftriaxone, or aminopenicillins, such as ampicillin), or shigellosis.

The combination therapy may be administered as a simultaneous or sequential regimen. When administered sequentially, the combination may be administered in two or more administrations.

Co-administration of a peptide conjugate of the invention with one or more other active therapeutic agents generally refers to simultaneous or sequential administration of a peptide conjugate of the invention and one or more other active therapeutic agents, such that therapeutically effective amounts of the peptide conjugate of the invention and one or more other active therapeutic agents are both present in the body of the patient.

Co-administration includes administration of unit dosages of the peptide conjugates of the invention before or after administration of unit dosages of one or more other active therapeutic agents, for example, administration of the peptide conjugates of the invention within seconds, minutes, or hours of the administration of one or more other active therapeutic agents and/or as part of the same treatment regimen. For example, a unit dose of a peptide conjugate of the invention can be administered first, followed within seconds or minutes or days by administration of a unit dose of one or more other active therapeutic agents. Alternatively, a unit dose of one or more other therapeutic agents can be administered first, followed by administration of a unit dose of a peptide conjugate of the invention within seconds or minutes or days. In some cases, it may be desirable to administer a unit dose of a peptide conjugate of the invention first, followed, after a period of hours (e.g., 1-12 hours), by administration of a unit dose of one or more other active therapeutic agents. In other cases, it may be desirable to administer a unit dose of one or more other active therapeutic agents first, followed, after a period of hours (e.g., 1-12 hours), by administration of a unit dose of a peptide conjugate of the invention.

The combination therapy may provide “synergy” and “synergistic,” i.e., the effect achieved when the active ingredients used together is greater than the sum of the effects that results from using the peptide conjugates separately.

As used herein, the words “a” and “an” are meant to include one or more unless otherwise specified. For example, the term “an agent” encompasses both a single agent and a combination of two or more agents.

The term “treating” or “treatment” as used herein covers the treatment of the disease or condition of interest (e.g., a viral infection) in a mammal, preferably a human, having the disease or condition of interest, and includes, for example: preventing or delaying the onset of the disease or condition from occurring in a mammal, in particular, when such mammal is at risk of developing the disease but has not yet become symptomatic and/or been diagnosed as having it; inhibiting the disease or condition, i.e., arresting its development; relieving the disease or condition, i.e., causing regression of the disease or condition; and/or stabilizing the disease or condition. Treatment includes ameliorating or lessening the severity of symptoms of the disease or condition, and/or inhibition of further progression or worsening of those symptoms. Treatment also includes shortening the time course and/or severity of a disease or condition compared to the expected or historical time course and/or severity of the disease.

As used herein the terms “preventing,” means causing the clinical symptoms of a disease or condition not to develop and includes inhibiting the onset of a viral infection in a subject that may be exposed to or predisposed to the viral infection but does not yet experience or display symptoms of the infection.

An “effective amount” or a “therapeutically effective amount” of a peptide conjugate or composition described herein refers to an amount of the peptide conjugate that is sufficient to achieve a specific effect or result, and/or prevents or treats the disease or condition and/or the symptoms therefore, for example, alleviating, in whole or in part, symptoms associated with the disorder or condition, or halts or slows further progression or worsening of those symptoms, or prevents or provides prophylaxis for the disorder or condition. The “effective amount” and “therapeutically effective amount” includes specifically an anti-viral amount of a peptide conjugate of the invention (alone or in combination with another active agent) or the composition described herein.

While this invention has been particularly shown and described with references to preferred embodiments thereof, it will be understood by those skilled in the art that various changes in form and details may be made therein without departing from the scope of the invention encompassed by the appended claims.

### Examples

### SARS-COV-2 Animal Model

KU LEUVEN R&D has developed and validated a SARS-COV-2 Syrian Golden hamster infection model. See Boudewijns, et al. “STAT2 signaling restricts viral dissemination but drives severe pneumonia in SARS-COV-2 infected hamsters.” Nat Commun. 2020 Nov. 17; 11 (1): 5838. Sanchez et al, “A single-dose live-attenuated YF17D-vectored 1 SARS-COV2 vaccine candidate.” Nature 2020. This model is suitable for the evaluation of the potential antiviral activity and selectivity of novel therapeutics. See Kaptein, et al, “Favipiravir at high doses has potent antiviral activity in SARS-COV-2-infected hamsters, whereas hydroxychloroquine lacks activity.” Proc Natl Acad Sci USA 2020 Oct. 9; 202014441. Abdelnabi, et al, “The oral protease inhibitor (PF-07321332) protects Syrian hamsters against infection with SARS-COV-2 variants of concern.” bioRxiv 2021.11.04.467077.

### SARS-COV-2

Delta B.1.617.2 (derived from hCoV-19/Belgium/rega-7214/2021; EPI_ISL_2425097; 2021 Apr. 20) was originally isolated in-house from nasopharyngeal swabs taken from a traveler returning to Belgium (baseline surveillance). Abdelnabi, et al, “The oral protease inhibitor (PF-07321332) protects Syrian hamsters against infection with SARS-COV-2 variants of concern.” bioRxiv 2021.11.04.467077. The variant was subjected to sequencing on a MinION platform (Oxford Nanopore) directly from the nasopharyngeal swabs; passage 2 virus on Vero E6 cells was used for the study described herein. The titer of the virus stock was determined by end-point dilution by the Reed and Muench method. Reed, L. J. et al, “A simple method of estimating fifty percent endpoints”. Am. J. Hyg. 1938, 27, 493-497. Live virus-related work was conducted in the high-containment A3 and BSL3+facilities of the KU Leuven Rega Institute (3CAPS) under licenses AMV 30112018 SBB 219 2018 0892 and AMV 23102017 SBB 219 20170589 according to institutional guidelines.

### Cells

Vero E6 cells (African green monkey kidney, ATCC CRL-1586) are cultured in minimal essential medium (Gibco) supplemented with 10% fetal bovine serum (Integro), 1% L-glutamine (Gibco) and 1% bicarbonate (Gibco). A549-Dual™ hACE2-TMPRSS2 cells obtained by Invitrogen (Cat. a549d-cov2r) are cultured in DMEM 10% FCS (Hyclone) supplemented with 10 μg/ml blasticidin (Invivigen, ant-bl-05), 100 μg/ml hygromycin (Invivogen, ant-hg-1), 0.5 μg/ml puromycin (Invivogen, ant-pr-1) and 100 μg/ml zeocin (Invivogen, ant-zn-05). End-point titrations were performed with medium containing 2% fetal bovine serum instead of 10% and no antibiotics.

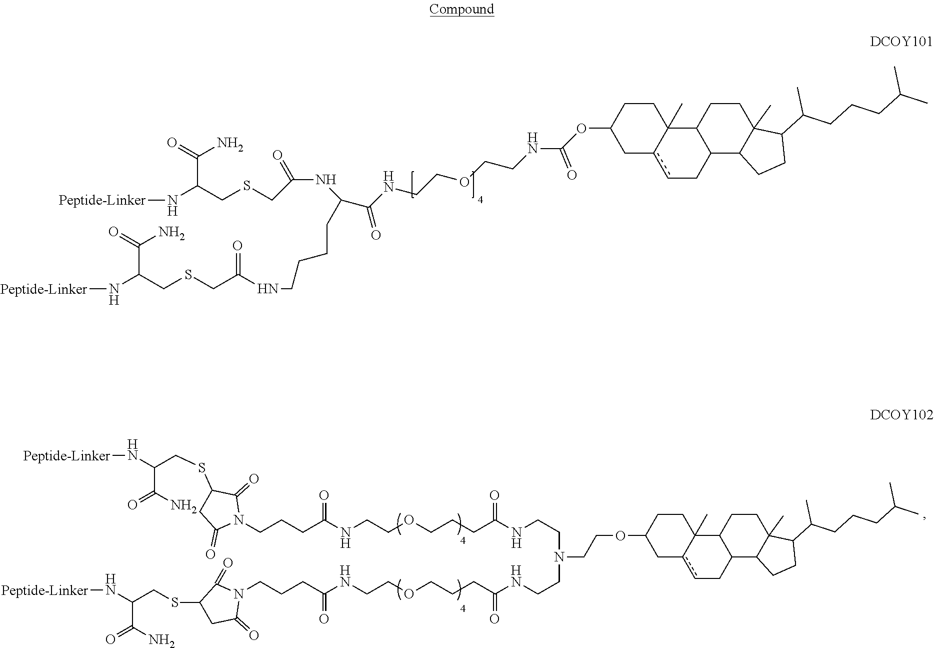

and each Peptide-Linker is

(SEQ ID NO. 5)
H2N-DISGINASVVNIQKEIDRLNEVAKNLNESLIDLQELGSGSG

### Formulation

Vehicle=280 mM D-mannitol in 0.1×PBS at pH 6
Drug=20 mg/mL DCOY 101, 280 mM D-mannitol in 0.1×PBS at pH 6; or
Drug=20 mg/mL DCOY 102, 280 mM D-mannitol in 0.1×PBS at pH 6

Material is stored upright at refrigerated temperature (+2° C. to +8° C.) upon receipt and for the duration of the study. The solution is mixed using gently pipette prior to treatment to ensure uniform mixture.

### SARS-COV-2 Infection Model in Hamsters

36 wild-type female Syrian Golden hamsters (Mesocricetus auratus) of 6-8 weeks old are purchased from Janvier Laboratories and housed per two in ventilated isolator cages (IsoCage N Biocontainment System, Tecniplast) with ad libitum access to food and water and cage enrichment (wood block). The animals are acclimated for 4 days prior to study start. Housing conditions and experimental procedures were approved by the ethics committee of animal experimentation of KU Leuven (license P065-2020). On Day 0, animals are separated as 18 Donor (index) that are infected on that day and 18 Contact hamsters (that are co-housed with infected donor animals on day 1 morning as pairs). On day 0, the donor hamsters are anesthetized intraperitoneally Ketamin (60 mg/kg)+Xylazin (6 mg/kg) and inoculated intranasally with 50 μL containing 1×10⁴ TCID₅₀ SARS-COV-2 delta variant.

### Treatment Regimen

Treatment groups of this study consist of two pre-treatment (PrEP) groups (vehicle and DCOY101 (or DCOY102) (start 24h before infection)) and one post-treatment (PEP) DCOY101 (or DCOY102) group (start 8h post-infection, pi), where each group is further divided into Donor group (intranasally infected) and Contant group (uninfected). Therefore, there are 6 groups, and each group consists of 6 hamsters, where Groups 1, 3, and 5 are Donor groups and Groups 2, 4, and 6 are Contact groups (See Table 2). Starting from Day 1, each hamster in Group 1 is co-housed with one hamster in Group 2, and so are Group 3 and 4, and Group 5 and 6. For treatment, animals are anesthetized intraperitoneally Ketamin (60 mg/kg)+Xylazin (6 mg/kg) and inoculated intranasally once daily with 50 μL (25 μL per nare; fixed amount administered without weight adjustment) of the corresponding treatment and treatment are continued until day 3 pi for all treatment group. Hamsters are monitored every day for appearance, behavior and weight. At day 4 pi, all hamsters are euthanized by i.p. injection of 500 μL Dolethal (200 mg/mL sodium pentobarbital, Vétoquinol SA). Lungs are collected and viral RNA and infectious virus are quantified by RT-qPCR and end-point virus titration, respectively. The whole left lungs are collected in 4% Formaldehyde for fixation then subjected to histopathological staining with H&E and scoring by certified pathologist from KU Leuven. Optionally, throat swabs are collected on day 2, 3 and 4 pi and subjected for RT-qPCR quantification. One treatment regimen is depicted in FIG. 1.

### Body Weights

All hamsters are individually weighed and recorded at the following time points:
Prior to PreP treatment on Day-1
Once daily on Study Days 0 to 4

The percent change in body weight from the Day-1 through D4 body weight (baseline) are calculated for each hamster.

TABLE 2
Overview of the study design
Animal groups for transmission study

| | | Dose | Dosing | Start of | Number of |
|---|---|---|---|---|---|
| Groups | Compound | mg/kg | regimen | treatment | hamsters |
| 1 | Index (Donor), | 10 | 5 days QD | 24 h prior to | 6 |
| | vehicle | | intranasal | infection | |
| 2 | Contacts | | | | |
| | (sentinels) for | / | / | / | 6 |
| | Group1 | | | | |
| 3 | Index (Donor), | 10 | 5 days QD | 24 h prior to | 6 |
| | DCOY101 (or | | intranasal | infection | |
| | DCOY102) | | | | |
| | PrEP | | | | |
| 4 | Contacts | / | / | / | 6 |
| | (sentinels) for | | | | |
| | Group2 | | | | |
| 5 | Index (Donor), | 10 | 4 days QD | 8 h post- | 6 |
| | DCOY101 or | | intranasal | infection | |
| | (DCOY102) | | | | |
| | PEP | | | | |
| 6 | Contacts | / | / | / | 6 |
| | (sentinels) for | | | | |
| | Group3 | | | | |

Overview of manipulations

| Day −1 | Day 0 | Day 1 | Day 2 | Day 3 | Day 4 |
|---|---|---|---|---|---|
| Morning | Morning | Morning | Morning | Morning | Morning |
| Groups 1 | Separate | Groups 1, 3 | Groups 1, 3 | Groups 1, 3 | Groups 1-6 |
| and 3: | donor and | and 5: | and 5: | and 5: | weight |
| weight | contacts | weight | weight | weight | sacrifice |
| treatment | infect all | treatment | treatment | treatment | lung |
| | donors | Co-housing | collect | | collections |
| | Groups 1 | donor with | throat swabs | | collect throat |
| | and 3: | contact | (optional) | | swabs |
| | weight | animals | | | (optional) |
| | treatment | | | | |
| | Afternoon | | | | |
| | (8 hpi) | | | | |
| | Group 5 | | | | |
| | weight | | | | |
| | treatment | | | | |

Donor will be infected intranasally with 1×10⁴ TCID₅₀ Delta variant (in 50 μL volume). On Lung samples for qRT-PCR, Titration and histopathology analysis

### SARS-COV-2 RT-qPCR

Hamster lung tissues from all animals on study are collected after sacrifice and homogenized using bead disruption (Precellys) in 350 μL TRK lysis buffer (E.Z.N.A.® Total RNA Kit, Omega Bio-tek) and centrifuged (10.000 rpm, 5 min) to pellet the cell debris. RNA are extracted according to the manufacturer's instructions. Of 50 μL eluate, 4 μL are used as a template in RT-qPCR reactions. RT-qPCR are performed on a LightCycler96 platform (Roche) using the iTaq Universal Probes One-Step RT-qPCR kit (BioRad) with N₂ primers and probes targeting the nucleocapsid. Standards of SARS-COV-2 cDNA (IDT) are used to express viral genome copies per mg tissue.

### Throat Swabs Collection and Quantification

For collection of throat swabs from all animals on day 2 and day 4 pi, swabs with mini tips (VWR, Cat. No. 710-0464) are toughly rubbed into the throat to obtain the specimen and vortexed immediately for <1 min in 300 μl DNA/RNA shield. The long ends are cut and samples are centrifuged at 5000×g for 5 min at room temperature, the swabs are removed and samples are stored at RT until RNA extraction with NucleoSpin RNA kit and quantification by qRT-PCR as described before.

### End-Point Virus Titrations

Lung tissues from all animals in the study are homogenized using bead disruption (Precellys) in 350 μL minimal essential medium and centrifuged (10,000 rpm, 5 min, 4° C.) to pellet the cell debris. To quantify infectious SARS-COV-2 particles, endpoint titrations are performed on confluent A549-Dual™ hACE2-TMPRSS2 cells in 96-well plates. Viral titers are calculated by the Reed and Muench method using the Lindenbach calculator and are expressed as 50% tissue culture infectious dose (TCID₅₀) per mg tissue.

### Histology

For histological examination, the lungs are fixed overnight in 4% formaldehyde and transferred to Translational Cell and Tissue Research-Histopathological Services of KU Leuven. The fixed samples are processed overnight in the Spin Tissue Processor 120 (STP120; Epredia) followed by tissue embedding (TES99 Medite; VWR). 5 um sections are made using a semi-automatic microtome (Thermo Fisher/Leica). HE staining and coverslipping is performed respectively on the Autostained XL (Lecia) and the Robotic Coverslipper (Leica). Stained slides were scored blindly for lung damage by an expert pathologist (Prof. Birgit Weynand UZ Leuven). The scored parameters, to which a cumulative score of 1=mild, 2=moderate, to 3=severe was attributed, were the following: congestion, intra-alveolar hemorrhagic, apoptotic bodies in bronchus wall, necrotizing bronchiolitis, perivascular edema, bronchopneumonia, perivascular inflammation, peribronchial inflammation and vasculitis.

### Statistics

GraphPad Prism (GraphPad Software, Inc.) is used to perform statistical analysis. Statistical significance is determined using the non-parametric Mann Whitney U-test. P-values of <0.05 were considered significant.

### Transmission Prophylaxis Treatment

Transmission prophylaxis treatment study is also conducted following the experimental protocols as described above, except for that the treatment is administered to the Contact groups rather than the Donor (infected) groups.

### Animal Groups for Transmission Prophylaxis Study

| | | Dose | Dosing | Start of | Number of |
|---|---|---|---|---|---|
| Groups | Compound | mg/kg | regimen | treatment | hamsters |
| 1 | Infected | / | / | / | 12 |
| | (Donor) | | | | |
| 2 | Contacts for | 10 | 5 days QD | 24 h | 12 |
| | Group1, vehicle | | intranasal | prior to | |
| | | | | infection | |
| 3 | Infected | / | / | / | 12 |
| | (Donor) | | | | |
| 4 | Contacts for | 10 | 5 days QD | 24 h | 12 |
| | Group2, | | intranasal | prior to | |
| | DCOY101 (or | | | infection | |
| | DCOY102) | | | | |
| | PrEP | | | | |
| 5 | Uninfected | / | / | / | 12 |
| | Mock | | | | |

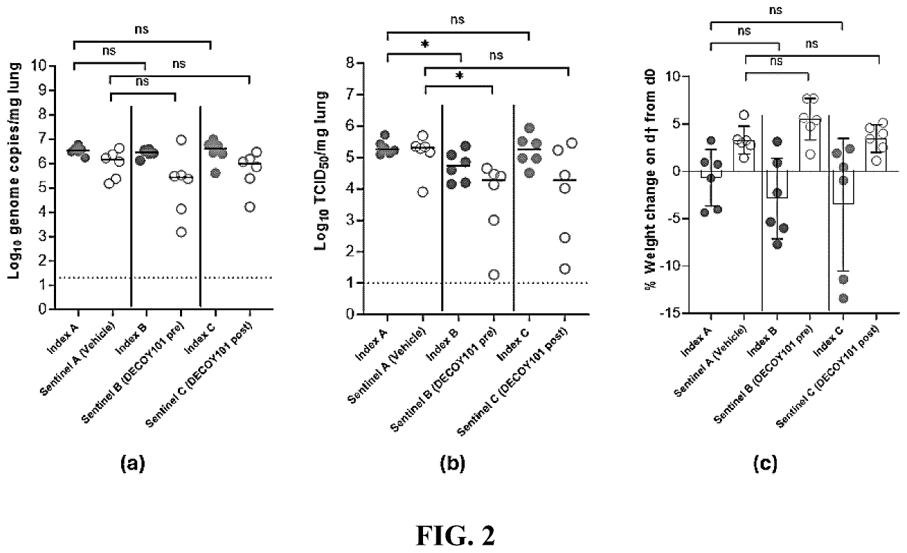

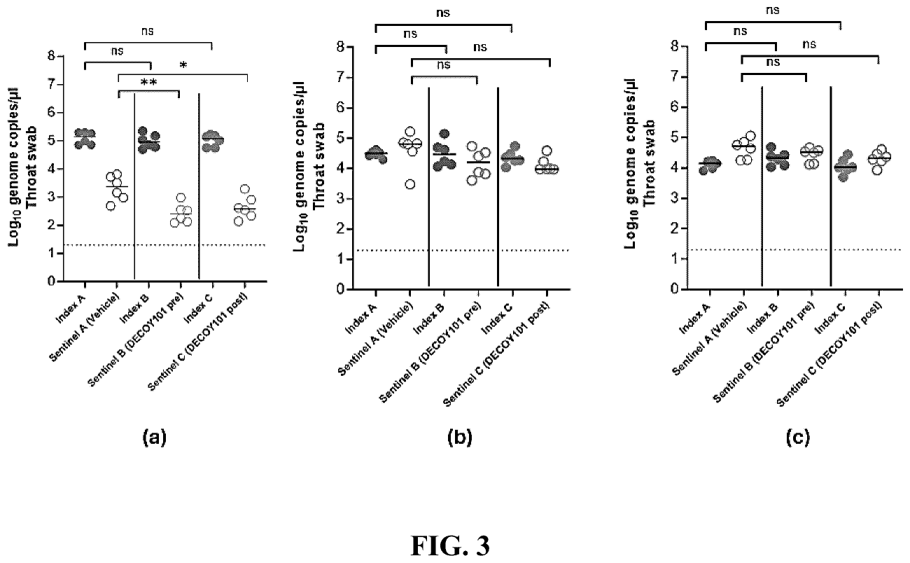

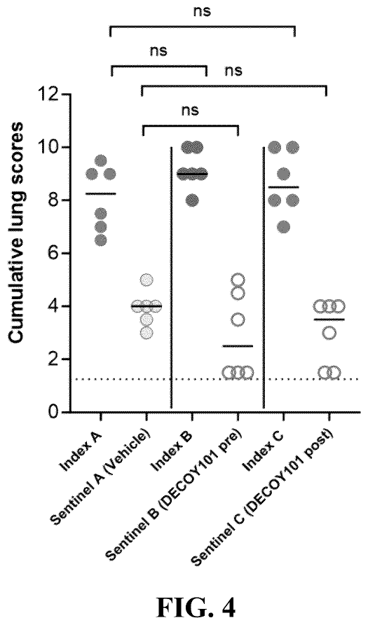

The transmission study as designed here showed evidence of prevention of transmission, as evidenced by a significant reduction in live virus in the DCOY101 PreP group. 10 mg/kg DCOY101 PreP showed a significant decrease in infectious virus in the lungs when compared to vehicle. FIG. 2(a) a terminal measurement of RT-qPCR demonstrated a mild trend of reduced viral genomes in DCOY101 Prep but results not significant. 2 (b) the terminal measurement of TCID₅₀ demonstrated a trend of reduced live virus observed in both DCOY101 Prep and PEP, with Prep results more significant than PEP. 2(c) showed the body weight change measured from day 1 of experiment to the day the animals were euthanized. From the efficacy evaluation based on throat swabs, as shown in FIG. 3, 10 mg/kg DCOY101 PreP and PEP on day 2 showed an impact on number of viral genomes (live & dead) present in the lung. As shown in FIG. 4, the lung histology results showed a decrease in histological measurement of lung disease associated with SARS-COV-2 infection in DCOY101 PrEP as compared to vehicle.

### Paramyxovirus

Table 3 and Table 4 provide the HR1 (HRN) protein receptor sequences and native HR2 (HRC) ligand sequences for Measles, Nipah and human parainfluenza virus (HPIV3), as well as three variants of the HPIV3 ligand.

TABLE 3
HR1 (Protein receptor) sequences

| | |
|---|---|
| Measles | MLNSQAIDNLRASLETTNQAIEAIRQAGQG |
| | MILAVQGVQDYINNELIPSMNQLSCDLIGQ |
| | (SEQ ID NO. 14) |
| Nipah | MKNADNINKLKSSIESTNEAVVKLQETAEK |
| | TVYVLTALQDYINTNLVPTIDKISCKQTEL |
| | (SEQ ID NO. 15) |
| HPIV3 | KQARSDIEKLKEAIRDTNKAVQSVQSSIGN |
| | LIVAIKSVQDYVNKEIVPSIARLGCEAAGL |
| | (SEQ ID NO. 16) |

TABLE 4
HR2 (Ligand) sequences

| | |
|---|---|
| Measles_ | ISLERLDVGTNLGNAIAKLEDAKELLES |
| native | SDQILRSM (SEQ ID NO. 12) |
| Nipah_ | VFTDKVDISSQISSMNQSLQQSKDYIKE |
| native | AQRLLDTV (SEQ ID NO. 13) |
| HPIV3_3001 | VALDPIDISIVLNKIKSDLEESKEWIRR |
| | SNKILDSI (SEQ ID NO. 9) |
| HPIV3_3002 | VALDPIDISIVLNKIKSQLEESKWEIRR |
| | SNKILDSI (SEQ ID NO. 10) |
| HPIV3_3003 | VALDPIDFSIVLNKIKSQLEESKWEIRR |
| | SNKILDSI (SEQ ID NO. 11) |
| HPIV3 native | VALDPIDISIELNKAKSDLEESKEWIRR |
| (HPIV3_3004) | SNQKLDSI (SEQ ID NO. 8) |

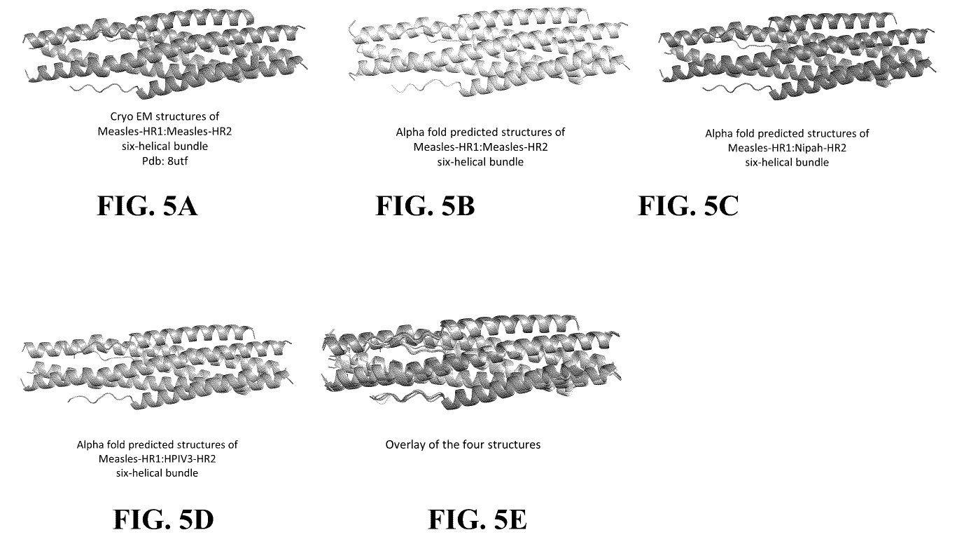

FIG. 5A through FIG. 5E show alpha fold predicted structures of viral HR1 and HR2 six helical bundles and an overlay of the bundles shown.

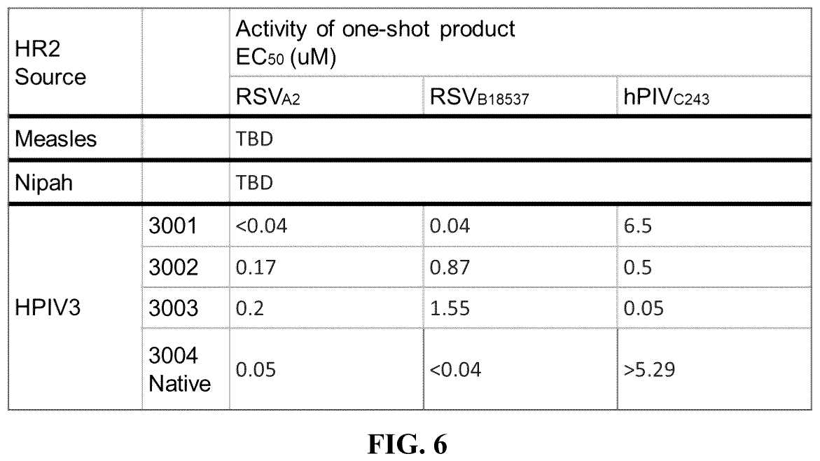

FIG. 6 shows the ligand binding (EC50 (uM) to certain viral proteins. The ligands are conjugated to cholesterol as disclosed in PCT/US24/31869, filed on May 31, 2024.

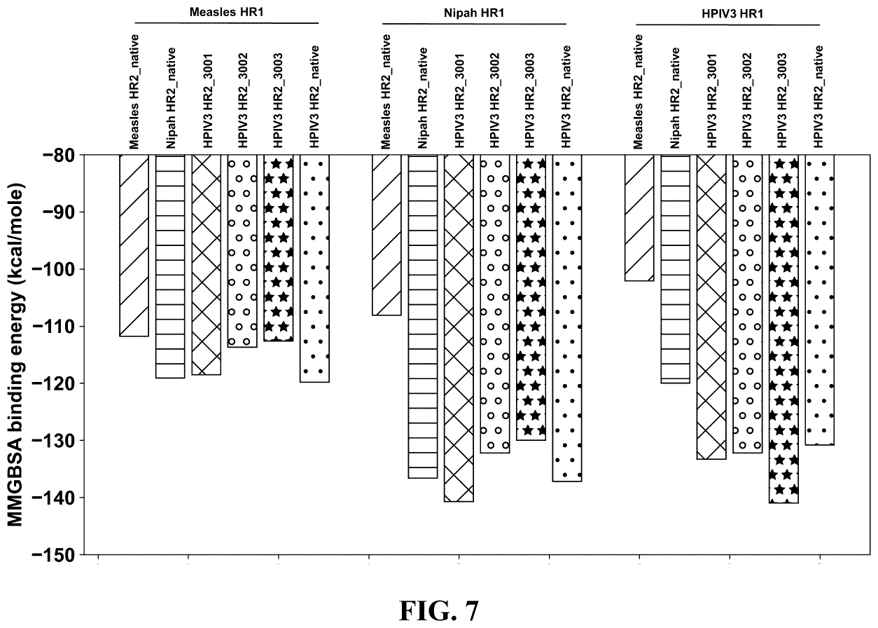

FIG. 7 shows the MMGBSA binding energy (kcal/mole) for each conjugated ligand with HR1 proteins of measles, Nipah and HPIV3.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims. It will also be understood that none of the embodiments described herein are mutually exclusive and may be combined in various ways without departing from the scope of the invention encompassed by the appended claims.

1. A method of preventing or reducing transmission of a SAR-Cov-2 variant from a first subject infected with the variant to a second subject who contacts the first subject and is uninfected with the variant before the contact, the method comprising administering to the first subject an effective amount of a peptide conjugate having the formula:
(Peptide-Linker)ₙ-B-Hydrophobic Moiety
wherein each Peptide is independently a therapeutic peptide or a targeting peptide, provided that at least one Peptide is a therapeutic peptide,
each Linker is independently an optional bivalent linking moiety,
B is a multivalent moiety,
Hydrophobic Moiety is a lipid or derivative thereof, and
n is an integer selected from 1, 2, 3 or more; and
wherein the variant comprises at least 5 mutations wherein the at least 5 mutations are independently in the spike protein S1 subunit or the S2 subunit or combinations thereof.

2. The method of claim 1, wherein the peptide conjugate is administered to the first subject before contact with the second subject and until the first subject tests negative for the variant.

3. The method of claim 1, wherein the peptide conjugate is administered to the first subject within 48 hours, 36 hours, 24 hours, 12 hours, or 8 hours of the discovery of the infection; or 48 hours, 36 hours, 24 hours, 12 hours, or 8 hours before the first subject contacts the second subject.

4. The method of claim 1, wherein the method further comprises an optional step of administering to the second subject an effective amount of the peptide conjugate.

5. The method of claim 4, wherein the peptide conjugate is administered to the second subject within 48 hours, 36 hours, 24 hours, 12 hours, or 8 hours before the first subject contacts the second subject; or within 48 hours, 36 hours, 24 hours, 12 hours, or 8 hours after the first subject contacts the second subject.

6. The method of claim 1, wherein the peptide conjugate is administered once two days, once a day, twice a day, three times a day, or four times a day; preferably once a day.

7. The method of claim 1, wherein the peptide conjugate is administered via an intranasal administration selected from an intranasal spray, an inhaler, a nebulizer, or any combination thereof.

8. The method of claim 1, wherein the contact occurs in an enclosed area.

9-12. (canceled)

13. The method of claim 1, wherein the at least 5 mutations are independently in N-Terminal domain (NTD), the receptor binding domain (RBD), the fusion peptide (FP) domain, the heptad repeat 1 (HR1) domain, or combinations thereof.

14. The method of claim 1, wherein the at least 5 mutations are independently selected from the group consisting of:
(i) at least 5 mutations from the SAR-Cov-2 Alpha variant;
(ii) at least 5 mutations from the SAR-Cov-2 Beta variant;
(iii) at least 5 mutations from the SAR-Cov-2 Delta variant; and
(iv) at least 5 mutations from the SAR-Cov-2 Omicron variant.

15. The method of claim 1, wherein the SARS-COV-2 comprises at least one variant selected from B.1.1.7 (Alpha), B.1.351 (Beta), P.1 (Gamma), B.1.617.2 (Delta), B.1.429/B.1.427 (Epsilon), B.1.617.1 (Kappa), B.1.525 (Eta), B.1.526 (Iota), P.3 (Theta), P.2 (Zeta), JN.1 (Pirola), B.1.621 (Mu), and B.1.1.529 (Omicron).

16. The method of claim 1, wherein the SARS-COV-2 comprises at least one variant selected from A.1-A.6, B.3-B.7, B.9, B.10, B.13-B.16, B.2, B. 1 lineage, P.1, P.2, P.3, and R.1.

17. The method of claim 14, wherein the B.1 lineage comprises at least one of (including, but not limited to), B.1, B.1.1, B.1.1.7, B.1.1.7 with E484K, B.1.2, B.1.5-B.1.72, B.1.9, B.1.13, B.1.22, B. 1.26, B.1.37, B.1.3-B.1.66, B.1.177, B.1.243, B.1.313, B. 1.351, B.1.427, B.1.429, B.1.525, B.1.526, B.1.526.1, B. 1.526.2, B.1.617, B. 1.617.1, B. 1.617.2, B.1.617.3, B.1.619, B.1.620, and B.1.621.

18. The method of claim 1, wherein Hydrophobic Moiety is selected from a cholesterol, a cholesterol ester, a phospholipid, and a sphingolipid.

19. (canceled)

20. The method of claim 1, wherein the B moiety comprises one or more thioether groups, one or more diamino acids, or both one or more thioether groups and one or more diamino acids.

21-22. (canceled)

23. The method of claim 1, wherein each Linker is independently an amino acid linker that comprises one or more glycine (G), serine (S), alanine (A), or any combination thereof and has about 2 to about 20 amino acids in length.

24. The method of claim 1, wherein the therapeutic peptide is a peptide inhibitor against the SARS-Cov-2 variant, and the targeting peptide is a receptor binding domain (RBD) binding peptide or an ACE2 targeting peptide.

25. The method of claim 1, wherein the therapeutic peptide is a HRC peptide of the SARS-Cov-2 variant, or an analog thereof.

26. The method of claim 1, wherein the therapeutic peptide is selected from:
Acₙ-DISGINASVVNIQKEIDRLNEVAKNLNESLIDLQEL (SEQ ID NO. 1), wherein n is 0 or 1;
(SEQ ID NO. 2)
dIdGdSdIdD NASVVNIQKEIDRLNEVAKNLNESLIDLQEL;
(SEQ ID NO. 3)
DISGINASVVNIQKEIDRLNEVAKNLNESLIDLQELGSGSG;
(SEQ ID NO. 4)
dIdGdSdIdD NASVVNIQKEIDRLNEVAKNLNESLIDLQELGSGSG;
(SEQ ID NO. 5)
H2N-DISGINASVVNIQKEIDRLNEVAKNLNESLIDLQELGSGSG;
(SEQ ID NO. 6)
Ac-dIdGdSdIdD NASVVNIQKEIDRLNEVAKNLNESLIDLQEL;
and
(SEQ ID NO. 7)
H2N-dIdGdSdIdD NASVVNIQKEIDRLNEVAKNLNESLIDLQEL.

27. (canceled)

28. The method of claim 1, wherein the peptide conjugate is any one of those in Table 1.

29-39. (canceled)
